SEQUENCE LISTING (8 sequences)

SEQ ID NO: 1 - protein, 124 aa, synthetic construct
  REGION 1..124: note = Synthetic Polypeptide
  REGION 1..124: note = MISC_FEATURE - Brazikumab heavy chain variable region amino acid sequence
  REGION 31..35: note = MISC_FEATURE - CDRH1
  REGION 50..67: note = MISC_FEATURE - CDRH2
  REGION 99..113: note = MISC_FEATURE - CDRH3
QVQLVESGGGVVQPGRSLRLSCAASGFTFSSYGMHWVRQAPGKGLEWVAVIWYDGSNEYYADSVKGRFTISRDNSKNTLYLQMNSLRAEDTAVYYCARDRGYTSSWYPDAFDIWGQGTMVTVSS

SEQ ID NO: 2 - protein, 111 aa, synthetic construct
  REGION 1..111: note = Synthetic Polypeptide
  REGION 1..111: note = MISC_FEATURE - Brazikumab light chain variable region amino acid sequence
  REGION 23..36: note = MISC_FEATURE - CDRL1
  REGION 52..58: note = MISC_FEATURE - CDRL2
  REGION 91..101: note = MISC_FEATURE - CDRL3
QSVLTQPPSVSGAPGQRVTISCTGSSSNTGAGYDVHWYQQVPGTAPKLLIYGSGNRPSGVPDRFSGSKSGTSASLAITGLQAEDEADYYCQSYDSSLSGWVFGGGTRLTVL

SEQ ID NO: 3 - protein, 5 aa, synthetic construct
  REGION 1..5: note = Synthetic Polypeptide
  REGION 1..5: note = MISC_FEATURE - Brazikumab CDRH1 amino acid sequence
SYGMH

SEQ ID NO: 4 - protein, 18 aa, synthetic construct
  REGION 1..18: note = Synthetic Polypeptide
  REGION 1..18: note = MISC_FEATURE - Brazikumab CDRH2 amino acid sequence
VIWYDGSNEYYADSVKGR

SEQ ID NO: 5 - protein, 15 aa, synthetic construct
  REGION 1..15: note = Synthetic Polypeptide
  REGION 1..15: note = MISC_FEATURE - Brazikumab CDRH3 amino acid sequence
DRGYTSSWYPDAFDI

SEQ ID NO: 6 - protein, 14 aa, synthetic construct
  REGION 1..14: note = Synthetic Polypeptide
  REGION 1..14: note = MISC_FEATURE - Brazikumab CDRL1 amino acid sequence
TGSSSNTGAGYDVH

SEQ ID NO: 7 - protein, 7 aa, synthetic construct
  REGION 1..7: note = Synthetic Polypeptide
  REGION 1..7: note = MISC_FEATURE - Brazikumab CDRL2 amino acid sequence
GSGNRPS

SEQ ID NO: 8 - protein, 11 aa, synthetic construct
  REGION 1..11: note = Synthetic Polypeptide
  REGION 1..11: note = MISC_FEATURE - Brazikumab CDRL3 amino acid sequence
QSYDSSLSGWV

(19) United States
(12) Patent Application Publication
(10) Pub. No.: US 2023/0122171 A1
(43) Pub. Date: Apr. 20, 2023

(54) Use of Brazikumab to Treat Crohn's Disease

(71) Applicant: ASTRAZENECA COLLABORATION VENTURES, LLC, Wilmington, DE (US)

(72) Inventors: Steven Shiff, Madison, NJ (US); Carl Gommoll, Robbinsville, NJ (US); Aparna Sahoo, Princeton, NJ (US)

(21) Appl. No.: 17/819,087

(22) Filed: Aug. 11, 2022

Related U.S. Application Data
(63) Continuation of application No. 16/999,470, filed on Aug. 21, 2020, now abandoned.
(60) Provisional application No. 62/890,017, filed on Aug. 21, 2019.

(51) Int. Cl. C07K 16/24; A61P 1/00; G01N 33/68

(52) U.S. Cl. CPC C07K 16/244; A61P 1/00; G01N 33/6869; A61K 2039/505

(57) ABSTRACT

The disclosure relates to products and methods for treating Crohn's disease. The products relate to antibodies that inhibit native human IL-23, but do not inhibit IL-12. The disclosure also relates to methods of selecting a subject amenable to IL-23 inhibition therapy to treat Crohn's disease as well as methods of identifying a patient sub-population amenable to such treatment.

### CROSS-REFERENCE TO RELATED APPLICATIONS

This application is a Continuation of U.S. application Ser. No. 16/999,470, filed Aug. 21, 2020, which claims the priority benefit under 35 U.S.C. § 119(e) of U.S. Provisional Patent Application No. 62/890,017, filed Aug. 21, 2019, which is incorporated herein by reference in their entirety.

### INCORPORATION BY REFERENCE OF MATERIALS SUBMITTED ELECTRONICALLY

This application contains, as a separate part of disclosure, a Sequence Listing in computer readable form (Filename: 54554B_Seqlisting.txt; Size: 21,057 bytes; Created: Aug. 9, 2022), which is incorporated herein by reference in its entirety.

### FIELD

The disclosure relates to products and methods for treating Crohn's disease. The products relate to antibodies that inhibit native human IL-23 but do not inhibit IL-12.

### BACKGROUND

IL-23, a member of the IL-12 family of cytokines, is a heterodimeric cytokine that potently induces pro-inflammatory cytokines. IL-23 is related to the heterodimeric cytokine Interleukin 12 (IL-12) both sharing a common p40 subunit. In IL-23, a unique p19 subunit is covalently bound to the p40 subunit. In IL-12, the unique subunit is p35 (Oppmann et al., Immunity, 2000, 13: 713-715). IL-23 is expressed by antigen presenting cells (such as dendritic cells and macrophages) in response to activation stimuli such as CD40 ligation, Toll-like receptor agonists and pathogens. IL-23 binds a heterodimeric receptor comprising an IL-12Rβ1 subunit (which is shared with the IL-12 receptor) and a unique receptor subunit, IL-23R.

IL-23 acts on activated and memory T cells and promotes survival and expansion of the T cell subset, Th17. Th17 cells produce proinflammatory cytokines, including IL-6, IL-17, TNFα, IL-22 and GM-CSF. IL-23 also acts on natural killer cells, dendritic cells and macrophages to induce pro-inflammatory cytokine expression. Unlike IL-23, IL-12 induces the differentiation of naïve CD4+ T cells into mature Th1 IFNγ-producing effector cells, and induces NK and cytotoxic T cell function by stimulating IFNγ production. Th1 cells driven by IL-12 were previously thought to be the pathogenic T cell subset in many autoimmune diseases; however, more recent animal studies in models of inflammatory bowel disease, psoriasis, inflammatory arthritis and multiple sclerosis, in which the individual contributions of IL-12 and IL-23 were evaluated, have firmly established that IL-23, not IL-12, is the key driver in autoimmune/inflammatory disease (Ahern et al., Immun. Rev. 2008 226:147-159; Cua et al., Nature 2003 421:744-748; Yago et al., Arthritis Res and Ther. 2007 9(5): R96). It is believed that IL-12 plays a critical role in the development of protective innate and adaptive immune responses to many intracellular pathogens and viruses and in tumor immune surveillance. See Kastelein, et al., Annual Review of Immunology, 2007, 25: 221-42; Liu, et al., Rheumatology, 2007, 46(8): 1266-73; Bowman et al., Current Opinion in Infectious Diseases, 2006 19:245-52; Fieschi and Casanova, Eur. J. Immunol. 2003 33:1461-4; Meeran et al., Mol. Cancer Ther. 2006 5: 825-32; Langowski et al., Nature 2006 442: 461-5. As such, IL-23 specific inhibition (sparing IL-12 or the shared p40 subunit) is expected to have a superior safety profile compared to dual inhibition of IL-12 and IL-23.

CD is an idiopathic, chronic transmural inflammatory disease that most commonly affects the distal ileum and colon, but may occur in any part of the gastrointestinal tract (Crohn's and Colitis Foundation of America 2012, Burger and Travis 2011, Rutgeerts 2003). CD may occur in any part of the gastrointestinal tract and has the potential for systemic and extraintestinal complications. Patients with CD have uncontrolled inflammation that causes direct damage to the intestinal mucosa. This inflammation is believed to result either from persistence of inflammatory stimulus, due to impaired gut barrier function, or from a dysregulated inflammatory response. CD occurs most commonly between the ages of 15 to 30 and 60 to 80, although persons of any age may be affected. Current treatment options for patients with moderately to severely active CD are usually guided by severity of disease, location, and presence of additional clinical complications, such as extraintestinal manifestations and malabsorption. The therapeutic options currently available are the “conventional treatments,” which include antibiotics, corticosteroids (CS), immunomodulators (azathioprine, 6-mercaptopurine, and methotrexate), and biologic treatments such as TNFα antagonists, integrin antagonists, and interleukin antagonists. Commonly used medical therapies include aminosalicylates, (including sulfasalazine and mesalamine), systemic CS, immunosuppressive agents (e.g., azathioprine, methotrexate), antibacterial agents, and biologic agents (e.g., adalimumab or Humira®, Abbvie, Inc, North Chicago, Ill.), infliximab (Remicade®, Janssen Biotech, Inc, USA), certolizumab (Cimzia®, UCB, Inc, Smyrna, Ga.), vedolizumab (Entyvio®, Takeda Pharmaceuticals America Inc, Deerfield, Ill.), and natalizumab (Tysabri®, Biogen Idec Inc, Cambridge, Mass.). Despite treatment with these agents, the residual morbidity and the complications of CD (e.g., intestinal obstruction and/or perforation, fistula formation, malnutrition) represent a burden of disease sufficient to warrant new therapies.

IL-23, a member of the IL-12 family of cytokines, is a heterodimeric cytokine consisting of two subunits: p40 and p19. The p40 subunit is shared by IL-12 and IL-23 as a common subunit and is targeted by inhibitors of IL-12/23 (e.g., ustekinumab and briakinumab). The main known effects of IL-23 are to drive the differentiation of T helper 17 cells, as well as macrophages, natural killer cells, dendritic cells, and innate lymphoid cells leading to up-regulation of IL-17, IL-22, TNFα, granulocyte-macrophage colony-stimulating factor, and IFNγ, and to down-regulation of IL-10 (Bettelli 2007).

Brazikumab is a human immunoglobulin that selectively binds to human IL-23 with high affinity and prevents IL-23 from interacting with the IL-23 receptor. The roles of IL-23 are believed to be important for the recruitment and activation of a range of inflammatory cells involved in IBD (CD and ulcerative colitis). In preclinical models and studies in patients, anti-IL-12/23 antibodies (eg, ustekinumab and briakinumab) have been shown to induce clinical responses in a variety of inflammatory diseases. Phase 2 data in participants with CD have demonstrated clinical efficacy of brazikumab comparable with that of antibodies targeting IL-12/23, suggesting that IL-23 activity may play an important, if not dominant, role in the inflammatory conditions under study. Thus, IL-23 blockade represents a novel mechanism to inhibit the inflammation and clinical symptoms associated with CD; specifically targeting IL-23 by brazikumab may offer a better benefit-risk profile compared with the IL-12/23 antibodies.

Targeting CD with brazikumab is supported by robust genetic and nonclinical data and by the demonstrated clinical efficacy of anti-IL-12/23p40 antibodies (ustekinumab and briakinumab) and anti-IL-23 p19 antibodies in CD (Mannon 2004, Sandborn 2012, Feagan 2016, Feagan 2017). Mice deficient in IL-23p19 are protected against experimental colitis, while mice deficient in IL-12p35 are not (Hue 2006, Yen 2006). Preclinical studies in several different animal models of IBD have demonstrated strong efficacy with IL-23-specific antagonism (Kullberg 2006, Uhlig 2006, Ahern 2008, IB Section 4.1).

In view of the foregoing observations, it is apparent that that there is a need for new modalities for the treatment of Crohn's disease that specifically target IL-23 without the potential risks associated with inhibition of IL-12. Further, a need continues to exist for methods of selecting subjects amenable to treatment of Crohn's disease by IL-23 inhibition and for methods of identifying patient sub-populations amenable to treatment of Crohn's disease by IL-23 inhibition.

### SUMMARY

Disclosed herein is an IL-23 blockade that provides a mechanism to inhibit the inflammation and reduce clinical symptoms associated with Crohn's disease (CD). The IL-23 blockade specifically inhibits IL-23 and does not inhibit IL-12, i.e., results in minimal (less than 1% IL-12 inhibition) or no inhibition of IL-12 activity following administration of brazikumab. In some embodiments, the IL-23 blockade specifically inhibits IL-23 and there is no inhibition of IL-12. Specifically targeting IL-23 with brazikumab is expected to offer a better benefit:risk profile compared with IL-12/23 antibodies.

Current treatment options for patients with moderately to severely active CD is usually guided by severity of disease, location, and presence of additional concomitant conditions and clinical complications such as extraintestinal manifestations and malabsorption. The therapeutic options currently available include ‘conventional treatments,’ which include antibiotics, CS, immunomodulators (azathioprine, 6-mercaptopurine, and methotrexate), and biologic treatments such as TNFα antagonists, integrin antagonists, and IL-12 and IL-23 antagonists.

In one aspect, the disclosure provides a method of treating Crohn's disease in a subject in need thereof comprising intravenous administration of an anti-IL-23 antibody to the subject followed by subcutaneous administration of the anti-IL-23 antibody to the subject. In some embodiments, a biological sample of the subject has an IL-22 level of at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 pg/ml, such as wherein the IL-22 level is at least 9 pg/ml or wherein the IL-22 level is at least 50 pg/ml. In some embodiments, the subject receives a plurality of intravenous administrations of the anti-IL-23 antibody, a plurality of subcutaneous administrations of the anti-IL-23 antibody, or both. In some embodiments, intravenous administrations are delivered within 4 weeks of initiating treatment. In some embodiments, intravenous administrations are delivered on days 1, 29 and 57 of treatment. In some embodiments, subcutaneous administrations are delivered at least 12 weeks after initiating treatment. In some embodiments, the subcutaneous administrations are delivered on about day 85 and about every 4 weeks thereafter. In some embodiments, the subcutaneous administrations are delivered on day 85 and about every 4 weeks thereafter, such as wherein the subcutaneous administrations are delivered on day 85 and every 4 weeks thereafter.

This aspect of the disclosure also provides some embodiments wherein the anti-IL-23 antibody is administered in an amount and at an interval of: (a) 720-1440 mg on or about days 1, 29, and 57 delivered intravenously, followed by (b) about 240 mg delivered subcutaneously on or about day 85 and about every 4 weeks thereafter through at least week 48. In some embodiments, the anti-IL-23 antibody has: (a) a heavy chain variable region comprising Complementarity Determining Regions (CDRs) with the following amino acid sequences: (i) CDR1: SYGMH (SEQ ID NO:3), (ii) CDR2: VIWYDGSNEYYADSVKGR (SEQ ID NO:4), and (iii) CDR3: DRGYTSSWYPDAFDI (SEQ ID NO:5); and (b) a light chain variable region comprising CDRs with the following amino acid sequences: (i) CDR1: TGSSSNTGAGYDVH (SEQ ID NO:6), (ii) CDR2: GSGNRPS (SEQ ID NO:7), and (iii) CDR3: QSYDSSLSGWV (SEQ ID NO:8). The brazikumab heavy- and light-chain variable region amino acid sequences are presented in FIG. 2. In some embodiments, the anti-IL-23 antibody is Brazikumab. In some embodiments, 720-1440 mg of Brazikumab is administered on days 1, 29, and 57. In some embodiments, Brazikumab is administered intravenously on days 1, 29, and 57. In some embodiments, 1440 mg of Brazikumab is administered intravenously on days 1, 29 and 57. In some embodiments, 720 mg of Brazikumab is administered intravenously on days 1, 29 and 57. In some embodiments, 240 mg of Brazikumab is administered subcutaneously on or about day 85 and about every 4 weeks thereafter through at least week 48, e.g., wherein 240 mg of Brazikumab is administered subcutaneously on day 85 and about every 4 weeks thereafter through at least week 48, such as by administering 240 mg of Brazikumab subcutaneously on day 85 and every 4 weeks thereafter through at least week 48. In some embodiments, 240 mg of Brazikumab is administered subcutaneously on day 85 and every 4 weeks thereafter through week 48-52. In some embodiments, 240 mg of Brazikumab is administered subcutaneously on day 85 and every 4 weeks thereafter through week 48.

Another aspect of the disclosure is drawn to a method of selecting a subject amenable to treatment of Crohn's disease comprising (a) obtaining a biological sample from the subject; (b) measuring the level of IL-22 in the sample; (c) comparing the level of IL-22 in the sample to the level of IL-22 in a control; and (d) selecting the subject as amenable to treatment of Crohn's disease if the level of IL-22 is higher in the sample than in the control. In some embodiments, the IL-22 level is at least about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 pg/ml, such as wherein the IL-22 level is at least about 9 pg/ml, or wherein the IL-22 level is at least about 50 pg/ml, or wherein the IL-22 level is at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 pg/ml, or wherein the IL-22 level is at least 9 pg/ml, or wherein the IL-22 level is at least 50 pg/ml. In some embodiments, the control is a biological sample from a healthy subject. As noted above, some embodiments provide for a control that has a threshold level of IL-22 associated with subjects having Crohn's disease, as disclosed in U.S. Ser. No. 15/759,330, incorporated herein by reference. In one exemplary embodiment, the threshold level of IL-22 is about 15.6 picograms/ml. In some embodiments, the Crohn's disease (CD) is ileal CD and/or colonic CD. In some embodiments, the method further comprises a second measure of amenability to treatment of Crohn's disease, such as a blood test for anemia or infection, a stool test for infection, a breath test for hydrogen, a barium enema, an upper endoscopy, an upper gastrointestinal series, a colonoscopy, a sigmoidoscopy, a CT scan or an MRI. In some embodiments, the method further comprises administering to the subject an anti-IL-23 antibody, e.g., brazikumab, in an amount and at an interval of: (a) 720-1440 mg on about days 1, 29, and 57 delivered intravenously, followed by (b) about 240 mg delivered subcutaneously on or about day 85 and about every 4 weeks thereafter through at least week 48.

Another aspect of the disclosure is a method of identifying a subject as a member of a patient sub-population amenable to treatment for Crohn's disease comprising (a) obtaining a biological sample from the subject; (b) measuring the level of IL-22 in the sample; (c) comparing the level of IL-22 in the sample to the level of IL-22 in a control; and (d) identifying the subject as a member of the patient sub-population amenable to treatment for Crohn's disease if the level of IL-22 in the subject's sample is higher than in the control. In some embodiments, the IL-22 level is at least about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 pg/ml, such as wherein the IL-22 level is at least about 9 pg/ml, or wherein the IL-22 level is at least about 50 pg/ml, or wherein the IL-22 level is at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 pg/ml, or wherein the IL-22 level is at least 9 pg/ml, or wherein the IL-22 level is at least 50 pg/ml. In some embodiments, the control is a biological sample from a healthy subject. In some embodiments, the control is a threshold level of IL-22 associated with subjects having Crohn's disease. In some embodiments, the Crohn's disease (CD) is ileal CD and/or colonic CD. In some embodiments, the method further comprises a second measure of amenability to treatment of Crohn's disease, such as a blood test for anemia or infection, a stool test for infection, a breath test for hydrogen, a barium enema, an upper endoscopy, an upper gastrointestinal series, a colonoscopy, a sigmoidoscopy, a CT scan or an MRI. In some embodiments, the method further comprises administering to the subject an anti-IL-23 antibody, e.g., brazikumab, in an amount and at an interval of: (a) 720-1440 mg on about days 1, 29, and 57 delivered intravenously, followed by (b) about 240 mg delivered subcutaneously on about day 85 and about every 4 weeks thereafter through at least week 48.

Yet another aspect of the disclosure is an anti-IL-23 antibody for use in treating Crohn's disease in a subject in need thereof, wherein the anti-IL-23 antibody is administered in an amount and at an interval of: (a) 720-1440 mg on or about days 1, 29, and 57 delivered intravenously, followed by (b) about 240 mg delivered subcutaneously on or about day 85 and about every 4 weeks thereafter through at least week 48. In some embodiments, the anti-IL-23 antibody has: (a) a heavy chain variable region comprising Complementarity Determining Regions (CDRs) with the following amino acid sequences: (i) CDR1: SYGMH (SEQ ID NO:3), (ii) CDR2: VIWYDGSNEYYADSVKGR (SEQ ID NO:4), and (iii) CDR3: DRGYTSSWYPDAFDI (SEQ ID NO:5); and (b) a light chain variable region comprising CDRs with the following amino acid sequences: (i) CDR1: TGSSSNTGAGYDVH (SEQ ID NO:6), (ii) CDR2: GSGNRPS (SEQ ID NO:7), and (iii) CDR3: QSYDSSLSGWV (SEQ ID NO:8). In some embodiments, the anti-IL-23 antibody is Brazikumab. In some embodiments, 720-1440 mg of Brazikumab is administered on days 1, 29, and 57. In some embodiments, Brazikumab is administered intravenously on days 1, 29, and 57. In some embodiments, 1440 mg of Brazikumab is administered intravenously on days 1, 29 and 57. In some embodiments, 720 mg of Brazikumab is administered intravenously on days 1, 29 and 57. In some embodiments, 240 mg of Brazikumab is administered subcutaneously on or about day 85 and about every 4 weeks thereafter through at least week 48. In some embodiments, 240 mg of Brazikumab is administered subcutaneously on day 85 and about every 4 weeks thereafter through at least week 48. In some embodiments, 240 mg of Brazikumab is administered subcutaneously on day 85 and every 4 weeks thereafter through at least week 48. In some embodiments, 240 mg of Brazikumab is administered subcutaneously on day 85 and every 4 weeks thereafter through week 48-52. In some embodiments, 240 mg of Brazikumab is administered subcutaneously on day 85 and every 4 weeks thereafter through week 48.

Embodiments of the method are also contemplated wherein a plurality of intravenous infusions is administered. In some embodiments, the plurality of intravenous infusions each comprise the same quantity of anti-IL-23 antibody. Embodiments of the disclosure also exist wherein the anti-IL-23 antibody is administered subcutaneously. In some of these embodiments, the anti-IL-23 antibody is administered in a plurality of doses.

Other features and advantages of the disclosure will become apparent from the following detailed description, including the drawing. It should be understood, however, that the detailed description and the specific examples, while indicating embodiments, are provided for illustration only, because various changes and modifications within the spirit and scope of the disclosure will become apparent to those skilled in the art from the detailed description.

### BRIEF DESCRIPTION OF THE DRAWINGS

FIG. 1 provides a schematic description of the protocol used to treat subjects with Crohn's disease.

FIG. 2 presents the amino acid sequences of brazikumab heavy and light chain variable regions, which are presented as SEQ ID NOs:1 and 2, respectively. Underscored amino acid sequences identify the six complementarity determining regions, i.e., CDRH1 (SEQ ID NO:3), CDRH2 (SEQ ID NO:4), CDRH3 (SEQ ID NO:5), CDRL1 (SEQ ID NO:6), CDRL2 (SEQ ID NO:7), and CDRL3 (SEQ ID NO:8).

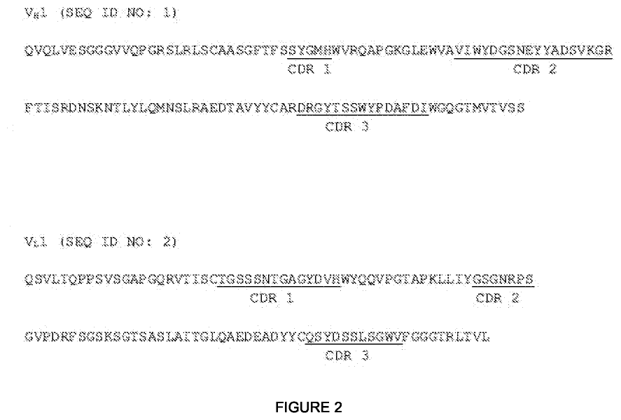

### DETAILED DESCRIPTION

The disclosure provides methods of treating, including ameliorating a symptom of, Crohn's disease (CD) by administering an effective amount of an anti-IL-23 antibody that inhibits an activity of IL-23 without inhibiting the activity of IL-12. In addition, the disclosure provides methods of identifying or selecting patients or patient populations afflicted with CD. The anti-IL-23 antibodies of the disclosure include all known forms of antibodies, provided that those antibody forms specifically bind and inhibit IL-23 without affecting the activity of IL-12. It is contemplated that the methods of the disclosure are well-suited for the treatment of patients with moderately to severely active Crohn's disease, typically as judged by a skilled clinician interpreting the results of a colonoscopy. The disclosed methods provide a cost-effective approach to bringing beneficial relief to those suffering from Crohn's disease.

The terms “treating”, and “treatment” and the like are used herein to generally mean obtaining a desired pharmacological, physiological or therapeutic effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease and/or its symptoms or conditions. The disclosure is directed towards treating a patient suffering from disease related to pathological inflammation. The present disclosure provides materials and methods for preventing, inhibiting, or relieving adverse effects attributed to pathological inflammation over long periods of time and/or are such as are caused by physiological response to inappropriate inflammation present in a biological system over a long period of time.

An anti-IL-23 antibody that does not inhibit IL-12, as used herein, means an anti-IL-23 antibody that results in minimal to no inhibition of IL-12 activity. An upper bound on minimal inhibition of IL-12 activity is less than 1% inhibition of IL-12 activity following administration of brazikumab.

In one aspect, the present disclosure provides methods of treating a subject. The method can, for example, have a generally beneficial effect on the subject, e.g., it can increase the subject's expected longevity. Alternatively, the method can, for example, treat, prevent, cure, relieve, or ameliorate (“treat”) a disease, disorder, condition, or illness (“a condition”). In one embodiment, the disclosure provides a method of treating a condition in a subject comprising administering the pharmaceutical composition comprising an IL-23-specific antibody to the subject, wherein the condition is treatable by reducing the activity (partially or fully) of IL-23 in the subject. Treating encompasses both therapeutic administration (i.e., administration when signs and symptoms of the disease or condition are apparent) as well prophylactic or maintenance therapy (i.e., administration when the disease or condition is quiescent), as well as treating to induce remission and/or maintain remission. Accordingly, the severity of the disease or condition can be reduced (partially, significantly or completely), or the signs and symptoms can be prevented or delayed (delayed onset, prolonged remission, or quiescence).

Among the conditions to be treated in accordance with the present disclosure are conditions in which IL-23 is associated with or plays a role in contributing to the underlying disease or disorder or otherwise contributes to a negative symptom. Such conditions include bowel inflammation, such as that characterizing Crohn's disease.

The term “efficacy” as used herein in the context of a dosage regimen refers to the effectiveness of a particular treatment regimen. Efficacy can be measured based on change in the course of a disease in response to an agent of the present disclosure. In one embodiment, an antigen-binding protein (for example, an anti-IL-23 antibody) is administered to the subject in an amount and for a time sufficient to induce an improvement, preferably a sustained improvement, in at least one indicator that reflects the severity of the disorder that is being treated. Various indicators that reflect the extent of the subject's illness, disease or condition may be assessed for determining whether the amount and time of the treatment is sufficient. Such indicators include, for example, clinically recognized indicators of disease severity, symptoms, or manifestations of the disorder in question.

In some embodiments according to the disclosure, an improvement is considered to be sustained if the subject exhibits the improvement on at least two occasions separated by two to four weeks. In another embodiment, an improvement is considered to be sustained if the subject exhibits the improvement on at least two occasions separated by two to four months; in a further embodiment, an improvement is considered to be sustained if the subject exhibits the improvement on at least two occasions separated by six to twelve months. The degree of improvement generally is determined by a physician, who may make this determination based on signs, symptoms, colonoscopies, biopsies, or other test results, and who may also employ questionnaires that are administered to the subject, such as quality-of-life questionnaires developed for a given disease such as Crohn's disease.

The IL-23 specific antibody may be administered to achieve an improvement in a subject's condition. Improvement may be indicated by a decrease in an index of disease activity, by amelioration of clinical symptoms, endoscopic improvement, or by any other measure of disease activity. In exemplary embodiments, the improvement in a subject's condition is histological improvement, determined from examination of biopsy samples. Histological improvement is at least one structural improvement in biological material (e.g., a cell), detected using any known form of microscopic analysis, including but not limited to infrared, electron, and light (e.g., confocal) microscopy. In addition, the improvement in a subject's condition is expected to be detectable using other techniques known in the art, including but not limited to Magnetic Resonance Enterography (MRE) in which an imaging test is used to evaluate gastrointestinal disorders, including inflammatory bowel diseases such as Crohn's disease.

Treatment of a subject with an IL-23 specific antibody may be given in an amount and/or at sufficient interval to achieve and/or maintain a certain quantity of IL-23-specific antibody per volume of serum using, for example, an assay as described herein. For example, the heterodimer specific antibody is given to achieve a serum concentration of 12.5 ng/ml to 1000 ng/ml. In one embodiment, the heterodimer-specific antibody is given to achieve a serum concentration of at least 12.5 ng/ml, 25 ng/ml, 50 ng/ml, 60 ng/ml, 70 ng/ml, 75 ng/ml, 80 ng/ml, 85 ng/ml, 90 ng/ml, 95 ng/ml, 100 ng/ml, 150 ng/ml, 200 ng/ml, 500 ng/ml, or 990 ng/ml. Those of skill in the art will understand that the amounts given here apply to a full-length antibody or immunoglobulin molecule; if an antigen-binding fragment thereof is used, the same molar serum concentration can be achieved although the weight per unit volume will differ from that given in a manner that can be calculated based on the molecular weight of the fragment and the full-length immunoglobulin.

It is understood that the methods of treating the diseases described herein would administer an effective amount of an anti-IL-23 antibody. Depending on the indication to be treated, a therapeutically effective amount is sufficient to cause a reduction in at least one symptom of the targeted pathological condition by at least about 5%, 10%, 15%, 20%, 25%, 30%, 35%, 40%, 45%, 50%, 55%, 60%, 65%, 70%, 75%, 80%, 85%, 90%, 95%, or more, relative to untreated subjects.

Administration and dosage regimens of an anti-IL-23 antibody can be adjusted to provide an effective amount for an optimum therapeutic response. For example, a single bolus can be administered, several divided doses can be administered over time or the dose can be proportionally reduced or increased as indicated by the exigencies of the therapeutic situation. The anti-IL-23 antibody may be administered by any suitable technique, including but not limited to, parenterally, topically, or by inhalation. If injected, the pharmaceutical composition can be administered, for example, via intra-articular, intravenous, intramuscular, intralesional, intraperitoneal or cutaneous routes (including intra-, trans- or sub- dermal, and subcutaneous), by bolus injection, or continuous infusion. In some embodiments, the pharmaceutical composition is administered by an intravenous route. In some embodiments the pharmaceutical composition is administered by a subcutaneous route. In further embodiments, the compositions are administered by oral, buccal, rectal, intratracheal, gastric, or intracranial routes. Localized administration, e.g., at a site of disease or injury is contemplated, for example, by enema or suppository for conditions involving the gastrointestinal tract. Also contemplated are transdermal delivery and sustained release from implants. Delivery by inhalation includes, for example, nasal or oral inhalation, use of a nebulizer, inhalation of the antagonist in aerosol form, and the like. Other alternatives include eyedrops; oral preparations including pills, syrups, lozenges or chewing gum; and topical preparations such as lotions, gels, sprays, and ointments.

Advantageously, IL-23 antibodies are administered in the form of a composition comprising one or more additional components such as a physiologically acceptable carrier, excipient or diluent. Optionally, the composition additionally comprises one or more physiologically active agents for combination therapy. A pharmaceutical composition may comprise an anti-IL-23 antibody together with one or more substances selected from the group consisting of a buffer, an antioxidant such as ascorbic acid, a low molecular weight polypeptide (such as those having fewer than 10 amino acids), a protein, an amino acid, a carbohydrate such as glucose, sucrose or dextrins, a chelating agent such as EDTA, glutathione, a stabilizer, and an excipient. In accordance with appropriate industry standards, preservatives such as benzyl alcohol may also be added. The composition may be formulated as a lyophilizate using appropriate excipient solutions (e.g., sucrose) as diluents. The anti-IL-23 antibody can be provided at a concentration of 50 to 200 mg/ml. Exemplary formulations useful for the present disclosure are those that include a glutamic acid, citric acid or acetic acid buffer at an appropriate pH, from 4.5 to 5.2, an excipient such as sucrose, glycine, proline, glycerol, and/or sorbitol at an appropriate concentration such as 1 to 20% (w/v), and a surfactant such as a non-ionic surfactant like polysorbate (polysorbate 20 or 80) or poloxamers (poloxamer 1888) at an appropriate concentration of 0.001% - 0.1% (w/v). Such formulations are disclosed in U.S. Pat. No. 6,171,586 and WIPO Published Applications Nos: WO20100027766 and WO2011088120. In some embodiments, the formulations comprise sodium acetate, sucrose and polysorbate 20. In some embodiments, the formulations comprise 70 mg/mL brazikumab, 10 mM sodium acetate, 9% (w/v) sucrose and 0.004% (w/v) polysorbate 20, at pH 5.2. Suitable components are nontoxic to recipients at the dosages and concentrations employed. Further examples of components that may be employed in pharmaceutical formulations are presented in any edition of Remington's Pharmaceutical Sciences including the 21^{st} Ed. (2005), Mack Publishing Company, Easton, Pa.

Kits for use by medical practitioners include an anti-IL-23 antibody and a label or other instructions for use in treating any of the conditions discussed herein. In one embodiment, the kit includes a sterile preparation of one or more IL-23 antigen-binding proteins, which may be in the form of a composition as disclosed above, and may be in one or more vials.

Particular embodiments of methods of the disclosure involve the use of an anti-IL-23 antibody and one or more additional IL-23 antagonists, as described in U.S. Pat. Nos. 7,491,391; 7,807,414; 7,872,102; 7,807,160; 8,362,212; 7,935,344; 7,790,862; U.S. Published Patent Application Nos. 2012282269, 20090123479; 20120128689; and 2012264917; and WIPO Publications WO1999/05280, WO2007/0244846, WO2007/027714, WO 2007/076524, WO2007/147019, WO2008/103473, WO 2008/103432, WO2009/043933, WO2009/082624 and WO 12/009760.

Also provided are IL-23 antibodies administered alone or in combination with other agents useful for treating Crohn's disease. Topical medications (e.g., steroids, coal tar, anthralin, Dead Sea salts, various natural oils, vitamin D3 and its analogs, sunshine, topical retinoids), phototherapy (e.g., ultraviolet light, photochemotherapy (PUVA)), and internal medications (e.g., methotrexate, systemic steroids). When multiple therapeutics are co-administered administered, dosages may be adjusted accordingly, as is recognized or known in the pertinent art.

In every case where a combination of molecules and/or other treatments is used, the individual molecule(s) and/or treatment(s) can be administered in any order, over any length of time that is effective, e.g., simultaneously, consecutively, or alternately. In one embodiment, the method of treatment comprises completing a first course of treatment with one molecule or other treatment before beginning a second course of treatment. The length of time between the end of the first course of treatment and beginning of the second course of treatment can be any length of time that allows the total course of therapy to be effective, e.g., seconds, minutes, hours, days, weeks, months, or even years.

The terms “polypeptide” or “protein” means a macromolecule having the amino acid sequence of a native protein, that is, a protein produced by a naturally occurring and non-recombinant cell; or it is produced by a genetically engineered or recombinant cell, and comprise molecules having the amino acid sequence of the native protein, or molecules having one or more deletions from, insertions to, and/or substitutions of the amino acid residues of the native sequence. The term also includes amino acid polymers in which one or more amino acids are chemical analogs of a corresponding naturally occurring amino acid and polymers. The terms “polypeptide” and “protein” encompass IL-23 antibodies and sequences that have one or more deletions from, additions to, and/or substitutions of the amino acid residues of the antigen-binding protein sequence. The term “polypeptide fragment” refers to a polypeptide that has an amino-terminal deletion, a carboxyl-terminal deletion, and/or an internal deletion as compared with the full-length native protein. Such fragments may also contain modified amino acids as compared with the native protein. In certain embodiments, fragments are about five to 500 amino acids long. For example, fragments may be at least 5, 6, 7, 8, 9, 10, 11, 12, 13, 14, 15, 16, 17, 18, 19, 20, 50, 70, 100, 110, 150, 200, 250, 300, 350, 400, or 450 amino acids long. Useful polypeptide fragments include immunologically functional fragments of antibodies, including binding domains. In the case of an anti-IL-23 antibody, useful fragments include but are not limited to one or more CDR regions, a variable domain of a heavy or light chain, a portion of an antibody chain, a portion of a variable region including less than three CDRs, an Fv, an scFv, a Fab, a Fab′, a F(ab′)2, and the like.

The term “isolated protein” refers to a protein, such as an antigen-binding protein (an example of which could be an antibody), that is purified from proteins or polypeptides or other contaminants that would interfere with its therapeutic, diagnostic, prophylactic, research or other use. As used herein, “substantially pure” means that the described species of molecule is the predominant species present, that is, on a molar basis it is more abundant than any other individual species in the same mixture. In certain embodiments, a substantially pure molecule is a composition wherein the object species comprises at least 50% (on a molar basis) of all macromolecular species present. In other embodiments, a substantially pure composition will comprise at least 80%, 85%, 90%, 95%, or 99% of all macromolecular species present in the composition. In certain embodiments, an essentially homogeneous substance has been purified to such a degree that contaminating species cannot be detected in the composition by conventional detection methods and thus the composition consists of a single detectable macromolecular species.

A “variant” of a polypeptide (e.g., an antigen-binding protein such as an antibody) comprises an amino acid sequence wherein one or more amino acid residues are inserted into, deleted from and/or substituted into the amino acid sequence relative to another polypeptide sequence. Variants include fusion proteins or chimeras. A “derivative” of a polypeptide is a polypeptide that has been chemically modified in some manner distinct from insertion, deletion, or substitution variants, e.g., via conjugation to another chemical moiety. Exemplary protein derivatives are forms of the protein that have been glycosylated, myristoylated, PEGylated, and the like.

The terms “naturally occurring” or “native” as used throughout the specification in connection with biological materials such as polypeptides, nucleic acids, host cells, and the like, refers to materials which are found in nature, such as native human IL-23. In certain aspects, recombinant antigen-binding proteins that bind native IL-23 are provided. In this context, a “recombinant protein” is a protein made using recombinant techniques, i.e., through the expression of a recombinant nucleic acid as described herein. Methods and techniques for the production of recombinant proteins are well known in the art.

The term “antibody” refers to an intact immunoglobulin of any isotype, and of any sub-isotype, or a fragment thereof that can compete with the intact antibody for specific binding to the target antigen, and includes, for instance, chimeric, humanized, fully human, and bispecific antibodies. An antibody as such is a species of an antigen-binding protein. Unless otherwise indicated, the term “antibody” includes, in addition to antibodies comprising two full-length heavy chains and two full-length light chains, derivatives, variants, fragments, and muteins thereof, examples of which are described below. An intact antibody generally will comprise at least two full-length heavy chains and two full-length light chains, but in some instances may include fewer chains such as antibodies naturally occurring in camelids, which may comprise only heavy chains. Antibodies may be derived solely from a single source, or may be “chimeric,” that is, different portions of the antibody may be derived from two different antibodies as described further below. The antigen-binding proteins, antibodies, or binding fragments may be produced in hybridomas, by recombinant DNA techniques, or by enzymatic or chemical cleavage of intact antibodies.

The term “functional fragment” (or simply “fragment”) of an antibody or immunoglobulin chain (heavy or light chain), as used herein, is an antigen-binding protein comprising a portion (regardless of how that portion is obtained or synthesized) of an antibody that lacks at least some of the amino acids present in a full-length chain but which is capable of specifically binding to an antigen. Such fragments are biologically active in that they bind specifically to the target antigen and can compete with other antigen-binding proteins, including intact antibodies, for specific binding to a given epitope. In one aspect, such a fragment will retain at least one complementarity determining region (CDR) present in the full-length light or heavy chain, and in some embodiments will comprise a single heavy chain and/or light chain or portion thereof. These biologically active fragments may be produced by recombinant DNA techniques, or may be produced by enzymatic or chemical cleavage of antigen-binding proteins, including intact antibodies. Fragments include, but are not limited to, immunologically functional fragments such as Fab, Fab′, F(ab′)2, Fv, domain antibodies and single-chain antibodies, and may be derived from any mammalian source, including but not limited to human, mouse, rat, goat, sheep, horse, cow, camelid or rabbit. It is contemplated further that a functional portion of the antigen-binding proteins disclosed herein, for example, one or more CDRs, could be covalently bound to a second protein or to a small molecule to create a therapeutic agent directed to a particular target in the body, possessing bifunctional therapeutic properties, or having a prolonged serum half-life.

An “antigen-binding protein” as used herein means a protein that specifically binds a specified target antigen; the antigen as provided herein is IL-23, particularly human IL-23, including native human IL-23. Antigen-binding proteins as provided herein interact with at least a portion of the unique p19 subunit of IL-23, detectably binding IL-23; but do not bind with any significance to IL-12 (e.g., the p40 and/or the p35 subunits of IL-12). As a consequence, the antigen-binding proteins provided herein are capable of affecting IL-23 activity without the potential risks that inhibition of IL-12 or the shared p40 subunit might incur. The antigen-binding proteins may affect the ability of IL-23 to interact with its receptor, for example by affecting binding to the receptor, such as by interfering with receptor association. In particular, such antigen-binding proteins totally or partially reduce, inhibit, interfere with or modulate one or more biological activities of IL-23. Such inhibition or neutralization disrupts a biological response in the presence of the antigen-binding protein compared to the response in the absence of the antigen-binding protein and can be determined using assays known in the art and described herein. Antigen-binding proteins provided herein inhibit IL-23-induced proinflammatory cytokine production, for example IL-23-induced IL-22 production in whole blood cells and IL-23-induced IFNγ expression in NK and whole blood cells. Reduction of biological activity can be about 20%, 30%, 40%, 50%, 60%, 70%, 80%, 85%, 90%, 91%, 92%, 93%, 94%,95%, 96%, 97% 98%, 99% or more.

Certain antigen-binding proteins described herein are antibodies, or are derived from antibodies. Such antigen-binding proteins include, but are not limited to, monoclonal antibodies, bispecific antibodies, minibodies, domain antibodies, synthetic antibodies, antibody mimetics, chimeric antibodies, humanized antibodies, human antibodies, antibody fusions, antibody conjugates, single chain antibodies, and fragments thereof, respectively. In some instances, the antigen-binding protein is an immunological fragment of an antibody (e.g., a Fab, a Fab′, a F(ab′)2, or a scFv).

Certain antigen-binding proteins that are provided may comprise one or more CDRs as described herein (e.g., 1, 2, 3, 4, 5, 6 or more CDRs). In some instances, the antigen-binding protein comprises (a) a polypeptide structure and (b) one or more CDRs that are inserted into and/or joined to the polypeptide structure. The polypeptide structure can take a variety of different forms. For example, it can be, or comprise, the framework of a naturally occurring antibody, or fragment or variant thereof, or may be completely synthetic in nature. Examples of various polypeptide structures are further described below.

An antigen-binding protein of the disclosure is said to “specifically bind” its target antigen when the dissociation equilibrium constant (K_{D}) is ≤10⁻⁸ M. The antigen-binding protein specifically binds antigen with “high affinity” when the K_{D} is ≤5×10⁻⁹M, and with “very high affinity” when the K_{D} is ≤5×10⁻¹⁰ M. In one embodiment the antigen-binding protein will bind to human IL-23 with a K_{D} of ≤5×10⁻¹² M, and in yet another embodiment it will bind with a K_{D} 5×10⁻¹³ M. In another embodiment of the invention, the antigen-binding protein has a K_{D} of ≤5×10⁻¹² M and a Koff of about ≤5×10⁻⁶ 1/s. In another embodiment, the Koff is ≤5×10⁻⁷1/s.

In embodiments where the antigen-binding protein is used for therapeutic applications, an antigen-binding protein can reduce, inhibit, interfere with or modulate one or more biological activities of IL-23, such as by inducing production of proinflammatory cytokines. IL-23 has many distinct biological effects, which can be measured in many different assays in different cell types; examples of such assays are known, see for example U.S. Published Patent Application No: 2013-0004501, the disclosure of which is incorporated by reference herein. Exemplary IL-23 antibodies are disclosed in U.S. Published Patent Application No: 2013-0004501.

As used herein, “brazikumab” (also known as AMG 139) refers to an intact brazikumab immunoglobulin or to an antigen-binding portion thereof that competes with the intact antibody for specific binding, unless otherwise specified. Brazikumab also includes antibodies (or fragments thereof) that are identical or similar to brazikumab in amino acid sequence, particularly in the variable regions, or in the CDRs thereof (however, variations in the constant regions are also contemplated). For example, a useful brazikumab polypeptide has an amino acid sequence that is 85%, 90%, 92%, 95%, 98%, 99% or 100% identical to that of a brazikumab polypeptide disclosed herein. In another embodiment, a useful polypeptide is between 80% 85%, 90%, 92%, 95%, 98%, 99% or 100% identical to brazikumab.

Brazikumab is a human antibody that specifically recognizes the native human IL-23 heterodimer, but does not bind with any significance to the human IL-12 heterodimer. Brazikumab inhibits IL-23-induced proinflammatory cytokine production. For example, IL-23-induced IL-22 production in whole blood cells and IL-23-induced IFNγ expression in NK and whole blood cells. In some embodiments, brazikumab is an isolated, IL-23-specific antigen-binding protein having a heavy chain variable region comprising CDRH1, CDRH2 and CDRH3 from SEQ ID NO:1, and a light chain variable region comprising CDRL1, CDRL2 and CDRL3 from SEQ ID NO:2. In some embodiments, brazikumab is an isolated, IL-23-specific antigen-binding protein wherein the heavy chain variable region is at least 90% identical to SEQ ID NO:1, and the light chain variable region is at least 90% identical to CDRL1, CDRL2 and CDRL3 from SEQ ID NO:2. See, WO 2011/056600, published May 11, 2011.

Where a range of values is provided, it is understood that each intervening value (to the tenth of the unit of the lower limit unless the context clearly dictates otherwise) between the upper and lower limit of that range, and any other stated or intervening value or smaller range in that stated range, is encompassed within the disclosure. The upper and lower limits of smaller ranges may independently be included in the smaller range, subject to any specifically excluded limit in the stated range. Where the stated range includes one or both of the limits, ranges excluding either both of those included limits are also included in the disclosure.

Unless otherwise defined herein, scientific and technical terms used in connection with the disclosure shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001), Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990). Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques are available for chemical syntheses, chemical analyses, pharmaceutical preparation, formulation, delivery, and treatment of patients.

In preclinical models and studies in patients, anti-IL-12/23 p40 antibodies (e.g., ustekinumab, which is approved for treatment of Crohn's disease and psoriasis, and briakinumab) and anti-IL-23p19 antibodies have been shown to induce clinical responses in Crohn's disease. Brazikumab, previously known as MEDI 2070 and AMG 139, is a human immunoglobulin that selectively binds to human interleukin-23 (IL-23) with high affinity and prevents IL-23 from interacting with the IL-23 receptor. The roles of IL-23 are believed to be important for the recruitment and activation of a range of inflammatory cells involved in inflammation. Brazikumab is a human, Chinese hamster ovary cell-derived, immunoglobulin G2 (IgG2) monoclonal antibody (mAb) consisting of 2 heavy chains of the IgG2 subclass and 2 light chains of the lambda subclass, which are covalently linked through disulfide bonds.

The nonclinical safety of brazikumab was evaluated in several studies with cynomolgus monkeys as the pharmacologically relevant species. In a safety pharmacology study, no brazikumab-related effects were noted on evaluated cardiovascular, respiratory, or neurobehavioral parameters after single intravenous (IV) administration of 300 mg/kg. In studies of 2 weeks, 3 months, and 6 months duration in cynomolgus monkeys, brazikumab was generally well tolerated when administered IV or subcutaneously (SC). Brazikumab administration at doses up to and including 300 mg/kg had no effect on in-life observations, peripheral blood immunophenotyping, or clinical and anatomic pathology, and no sex-related differences in exposure. In the 6-month toxicology study, administration of brazikumab to cynomolgus monkeys by SC injection at 30, 100, or 300 mg/kg once weekly for 26 weeks had no toxicologically significant effects on study parameters. Approximately 14% (4 of 28) of the brazikumab-treated animals developed binding anti-drug antibodies (ADA) during the dosing period and 25% (1 of 4) of animals at 300 mg/kg developed binding ADA in the recovery period. No neutralizing antibodies were detected in animals that tested positive for binding ADA, and binding ADA did not decrease brazikumab exposure. The no observed adverse effect level following 26 weekly SC doses of brazikumab was 300 mg/kg, the maximum dose tested, corresponding to a maximum serum drug concentration (Cₘₐₓ) of 5900 μg/mL and an area under the serum concentration versus time curve (AUC) of 32,100 μg·day/mL on Study Day 176.

Patients with IBD, specifically CD, have increased human IL-22 expression in the colonic tissue (Andoh 2005, Brand 2006), and serum IL-22 concentrations in patients with CD have been found to correlate strongly with disease activity. In a Phase 2a study evaluating the efficacy and safety of brazikumab in participants with moderately to severely active CD who failed treatment with an anti-TNFα agent, post-hoc analysis revealed a statistically significant treatment-by-Baseline for the serum IL-22 concentration interaction (p=0.04) in the logistic regression model used in the analysis of clinical response at Week 8, which suggested that the treatment effect at Week 8 differed by Baseline serum IL-22 concentration (Sands 2017). In addition, it was observed that the group of subjects receiving brazikumab had a substantial reduction in serum IL-22 concentration (81% reduction at Week 8 compared with Baseline), in contrast to the slight increase observed for the group of subjects receiving placebo (6% increase) (data on file).

Serum IL-22 concentrations are expected to have clinical relevance as a potential predictive BM for safe and effective use in patients with CD. The BM could potentially enable identification of a targeted subpopulation of patients who are most likely to experience a favorable clinical outcome with brazikumab treatment and reduce unnecessary exposure to subgroups not deriving optimal benefit. As disclosed herein, IL-22 is a suitable BM for Crohn's disease with threshold levels of at least about 9-50 pg/ml IL-22 (e.g., at least about 9 or at least about 50 pg/ml IL-22) serving to identify subjects with Crohn's disease suitable for treatment.

The nonclinical safety of brazikumab was evaluated in several studies with cynomolgus monkeys as the pharmacologically relevant species. In a safety pharmacology study, no brazikumab-related effects were noted on evaluated cardiovascular, respiratory, or neurobehavioral parameters after single IV administration of 300 mg/kg. In studies of 2 weeks, 3 months, and 6 months duration in cynomolgus monkeys, brazikumab was generally well tolerated when administered IV or SC. Brazikumab administration at doses up to and including 300 mg/kg had no effect on in-life observations, peripheral blood immunophenotyping, or clinical and anatomic pathology, and no sex-related differences in exposure. In the 6-month toxicology study, administration of brazikumab to cynomolgus monkeys by SC injection at 30, 100, or 300 mg/kg once weekly for 26 weeks had no toxicologically significant effects on study parameters. Approximately 14% (4 of 28) of the brazikumab-treated animals developed binding ADA during the dosing period and 25% (1 of 4) of animals at 300 mg/kg developed binding ADA in the recovery period. No neutralizing antibodies were detected in animals that tested positive for binding ADA, and binding ADA did not decrease brazikumab exposure. The no observed adverse effect level following 26 weekly SC doses of brazikumab was 300 mg/kg, the maximum dose tested, corresponding to a Cₘₐₓ of 5900 μg/mL and an AUC of 32,100 μg·day/mL on Study Day 176.

As described in further detail below, additional studies are expected to demonstrate the efficacy and safety of brazikumab in participants with moderately to severely active CD and demonstrate the clinical utility of serum IL-22 concentrations as a predictive BM to prospectively identify participants who are most likely to benefit from treatment with brazikumab.

Humira® (adalimumab) may be used as an active control for Stage 1 to provide internal evidence of assay sensitivity and as an active comparator for Stage 2 of this study. Humira® (adalimumab) could be chosen as the appropriate active comparator for this protocol because it is being used extensively to treat patients with CD and is considered an acceptable standard-of-care treatment in patients who have failed conventional non-biologic treatments [including antibiotics, CS, immunomodulators (azathioprine, 6-mercaptopurine, and methotrexate)]. Humira® is an acceptable alternate therapy in patients who have failed to demonstrate a response or have lost response to infliximab. Furthermore, practical considerations for participants were considered when selecting Humira® as the comparator such as the long-term SC dosing and the lower complexity needed to implement a double-dummy blinding strategy. Brazikumab (after the 3 IV infusion doses) and Humira® are both administered SC for the duration of the study. A detailed description of Humira® can be found in the manufacturer's prescribing information or local package insert (Humira® package insert).

Brazikumab is being developed as a treatment for CD to reduce intestinal inflammation and improve signs and symptoms in participants who have serum IL-22 concentrations at or above a pre-established cutoff (i.e., BM+), as determined in Stage 1 of the Phase 2b/3 design (noted below). This operationally seamless, Phase 2b/3 study design combines, within a single protocol, objectives that have traditionally been addressed in separate studies, and is intended to substantially reduce the time that would have occurred between the studies had they been conducted separately. The operationally seamless design allows for a confirmatory study to proceed after the Phase 2b study, but the data from the 2 studies are kept distinct. Stage 1 of this study represents the Phase 2b study, and Stage 2 is a Phase 3, confirmatory, marketing registration study. Stage 1 evaluates and establishes the clinical cutoff for serum IL-22 concentration as a potentially predictive in vitro companion diagnostic device, and the serum IL-22 concentration clinical cutoff is used to stratify participants for enrollment into Stage 2. The sponsor will commence Stage 2 after all participants are randomized into Stage 1 and have completed 12 weeks induction treatment and after the evaluation of the data from the Stage 1 interim analysis.

### EXAMPLES

### Example 1

The primary objectives of this study are to evaluate the efficacy and safety of brazikumab versus placebo (Stage 1) and versus Humira® (Stage 2) to achieve endoscopic response and clinical remission in participants with moderately to severely active CD who have inadequate response or are intolerant to conventional therapy (CS or immunomodulators; 6-mercaptopurine, azathioprine, methotrexate), are biological-treatment naive, or have demonstrated a successful response to prior biological treatment, or who have failed or were intolerant to biological treatment. However, participants who have failed (met the criteria for primary or secondary nonresponse to treatment) or were intolerant to treatment with Humira® will be excluded from participation.

This study will utilize an operationally seamless Phase 2b/3 clinical trial design as an alternative to the traditional drug development program of sequential, independent clinical trials. The confirmatory, operationally seamless Phase 2b/3 clinical trial has been developed to efficiently combine the Phase 2b and Phase 3 stages of drug development (Maca 2006). Furthermore, this operationally seamless Phase 2b/3 study design combines, within a single protocol, objectives that have traditionally been addressed in separate studies, and is intended to substantially reduce the time that would have occurred between the studies had they been conducted separately . This design allows for the confirmatory study (Stage 2) to proceed after Stage 1, but the data from the 2 studies are kept distinct. The sponsor will commence Stage 2 after all participants are randomized into Stage 1, have completed the Week 12 treatment period, and the data from the Stage 1 interim analysis has been fully evaluated.

This study is planned as a global, multicenter (approximately 400 sites), randomized, double-blind, double-dummy, active-and placebo-controlled, parallel-group, operationally seamless, Phase 2b/3, 52-week study. The protocol being implemented comprises 2 distinct study periods. Stage 1 is a Phase 2 study to evaluate the dose-response relationship to select IV brazikumab induction doses for continued development and to establish the serum IL-22 concentration clinical cutoff to stratify participants for enrollment into Stage 2. Participants in Stage 1 will be stratified by prior history of biologic use and current corticosteroid (CS) use. An interim analysis at Week 12 will be conducted to: (a) determine biomarker (BM) cutoff value of serum IL-22 concentration, (b) confirm number of brazikumab treatment arms for Stage 2, (c) confirm sample size for Stage 2 (to achieve endoscopic response and clinical remission at Week 12), and (d) confirm selection of patient-reported outcomes (PROs) for Stage 2 After Week 12, Stage 1 participants will continue with their assigned study group treatments through Week 52. Participants in Stage 1 are not eligible for enrollment in Stage 2. The interim analyses of Stage 1 will be performed after all randomized participants complete Week 12 and the data will be evaluated by the sponsor. Enrollment into Stage 2 (see below) will only commence at the direction of the sponsor after all go/no-go criteria have been evaluated and a satisfactory serum IL-22 concentration clinical cutoff has been established. In addition, the Week 52 analysis in Stage 1 will be used to determine if the sample size for Stage 2 needs to be adjusted. Lastly, based on the final analysis from Stage 1, a brazikumab IV treatment arm may be dropped from Stage 2.

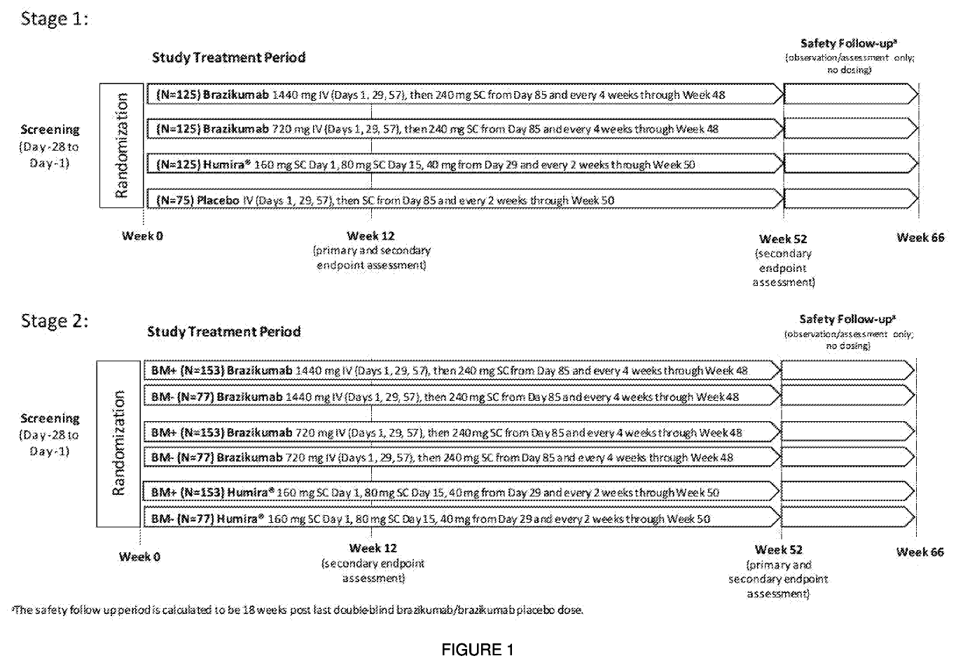

In Stage 2, a Phase 3 study is implemented to evaluate the safety and efficacy of brazikumab compared with Humira® in participants who are BM+ (serum IL-22 concentrations at or above a pre-established cutoff), and to validate the clinical utility of serum IL-22 concentration as a predictive biomarker (BM) for efficacy of brazikumab in a subset of participants with CD. Stage 2 Screening will be initiated upon completion of Stage 1, Week 12 interim analyses. The identification of a pre-specified serum IL-22 concentration cutoff in Stage 1 is used in the initiation of enrollment of Stage 2. The specified serum IL-22 concentration clinical cutoff is intended to represent a point where the sponsor can reliably identify the participants who are defined as being BM+ or BM− (serum IL-22 concentrations below a pre-established cutoff) for randomization into Stage 2 of the study. It is expected that brazikumab treatment will be more effective in the BM+ population compared to the BM− population. To confirm the appropriateness of restricting brazikumab use to a BM+ population, the primary objective and analysis will be based on the participants who are BM+. Data from participants who are BM− will serve as a reference to compare the clinical utility of serum IL-22 concentration in BM+ participants as a predictive BM for the efficacy of brazikumab. The treatment effect for brazikumab compared to Humira® is expected to be much smaller, if any, in the BM− participants. However, including BM− participants may provide an estimate of the effect in that population and may also potentially provide an overall risk—benefit assessment for brazikumab for the overall general population. The randomization stratification ratio of BM+ or BM− participants is planned to be 2:1 for all treatment groups; this ratio will be finalized upon review of the Stage 1 interim analysis. Investigators, participants, and sponsor personnel will remain blinded to BM+/BM− status for the duration of the study. Participants will also be stratified according to prior history of biologic use and current CS use. A study schematic is presented in FIG. 1, and Stage 1 objectives and endpoints are defined in Table 1.

TABLE 1
Stage 1 Objectives and Endpoints

| | |
|---|---|
| Stage 1 Objectives | Stage 1 Endpoints |

Primary

| | |
|---|---|
| To compare the efficacy of brazikumab with | Primary: Endoscopic response at Week 12: |
| that of placebo to achieve endoscopic | Minimum of 50% decrease from Baseline in |
| response and clinical remission at Week 12 | SES-CD total score |
| | Primary: Clinical remission at Week 12: |
| | Average daily LSF subscore of ≤3 as assessed |
| | on the CDAI LSF item AND |
| | Average daily AP subscore of ≤1 as assessed |
| | on the CDAI AP item |

Secondary

| | |
|---|---|
| To compare the efficacy of brazikumab | Secondary: Endoscopic response at both Week 12 |
| with that of placebo to achieve sustained | and Week 52 |
| endoscopic response and clinical | Secondary: Clinical remission at both Week 12 and |
| remission at both Week 12 and Week 52 | Week 52 |
| To compare the efficacy of brazikumab | Secondary: Endoscopic remission at Week 52 |
| with that of placebo in achieving | SES-CD total score of 0-2 OR |
| endoscopic remission and clinical | SES-CD total score of ≤4 and at least 2-point |
| remission at Week 52 | reduction from Baseline with no subscore > 1 |
| | Secondary: Clinical remission at Week 52 |
| To compare the efficacy of brazikumab | Secondary: Endoscopic response at Week 12 and |
| with that of placebo to achieve | endoscopic remission at Week 52 |
| endoscopic response at Week 12 and | Secondary: Clinical remission at both Week 12 and |
| endoscopic remission at Week 52 and | Week 52 |
| clinical remission at both Week 12 and | |
| Week 52 | |
| To compare the efficacy of brazikumab | Endoscopic response at Week 52 |
| with that of placebo to achieve | Clinical remission at Week 52 |
| endoscopic response and clinical | |
| remission at Week 52 | |
| To evaluate the efficacy of brazikumab to | Endoscopic response at Week 52 in participants who |
| achieve endoscopic response and clinical | are BM+ |
| remission at Week 52 in participants who | Clinical remission at Week 52 in participants who |
| have a BM value at or above a pre- | are BM+ |
| specified cut-off value | |
| To evaluate the PK and immunogenicity of | Population PK model of serum concentrations of |
| brazikumab in participants with CD | brazikumab and analysis for serum anti-brazikumab |
| | antibodies |
| To characterize the exposure-response | Exposure-response model linking primary endpoints |
| relationships of brazikumab | to metrics of model-predicted individual brazikumab |
| | exposures |
| To establish the serum IL-22 concentration | Exploration of relationship of Baseline serum IL-22 |
| Baseline clinical cutoff for its value in | concentration with efficacy of brazikumab at |
| predicting the efficacy of brazikumab | Week 12, and establishment of the serum IL-22 |
| | concentration clinical cutoff to stratify participants in |
| | Stage 2 |
| To evaluate the safety and tolerability of | AEs, clinical laboratory values, vital signs, physical |
| brazikumab in participants with CD | exams, ECGs |

Additional

| | |
|---|---|
| To compare the efficacy of brazikumab with | CS-free endoscopic remission at Week 52 |
| that of placebo to achieve CS-free (for the | CS-free clinical remission at Week 52 |
| last 12 weeks before the assessment at | |
| Week 52) endoscopic remission and clinical | |
| remission at Week 52 | |
| To compare the efficacy of brazikumab with | CS-free endoscopic remission at Week 52 for |
| that of placebo to achieve CS-free endoscopic | participants taking CS at Baseline |
| remission and clinical remission at Week 52 | CS-free clinical remission at Week 52 for |
| for participants taking CS at Baseline | participants taking CS at Baseline |
| To compare the efficacy of brazikumab with | Endoscopic remission at both Week 12 and Week 52 |
| that of placebo to achieve sustained | Clinical remission at both Week 12 and Week 52 |
| endoscopic remission at both Week 12 and | |
| Week 52 | |
| To compare the efficacy of brazikumab with | CS-free endoscopic response at Week 52 |
| that of placebo to achieve CS-free endoscopic | CS-free clinical remission at Week 52 |
| response and clinical remission at Week 52 | |
| To compare the efficacy of brazikumab with | Primary symptom remission at Week 12: |
| that of placebo to achieve primary symptom | For participants with Baseline LSF subscore |
| remission at Week 12 | of ≥5 and AP subscore < 2: Average daily LSF |
| | subscore of ≤3 AND no worsening of Baseline |
| | AP subscore as assessed on the CDAI OR |
| | For participants with Baseline AP subscore |
| | of ≥2 and LSF subscore < 5: Average daily AP |
| | subscore of ≤1 AND no worsening of Baseline |
| | LSF subscore as assessed on the CDAI |
| To compare the efficacy of brazikumab with | Primary symptom remission at Week 12 and |
| that of placebo to achieve sustained primary | Week 52 |
| symptom remission at Week 12 and Week 52 | |
| To compare the efficacy of brazikumab | Clinical response at Week 12 |
| with that of placebo to achieve clinical | Minimum 25% reduction in LSF subscore or AP |
| response at Week 12 | subscore from Baseline |
| To evaluate the impact of brazikumab on the | Change from Baseline at Week 12 and Week 52 in |
| signs and symptoms of CD at Week 12 and | signs and symptom scores (eg, LSF, AP, urgency, |
| Week 52 | fatigue) derived from the BSFS, NRS, CD-PRO, |
| | PGIS-CD, PIS-AP, PII-LBMF, PGIC-CD, and FACIT-F |
| To evaluate the impact of brazikumab on | Change from Baseline at Week 12 and at Week 52 in |
| HRQoL at Week 12 and Week 52 | IBDQ, SF-36, and EQ-5D-5L |
| To explore changes in signs and symptoms | Exploratory analysis of subscale scores from BSFS, |
| and HRQoL using a range of measures | CD-PRO, IBDQ, and Allergan-developed items |

Stage 2 objectives and endpoints are defined in Table 2.

TABLE 2
Stage 2 Objectives and Endpoints

| | |
|---|---|
| Stage 2 Objectives | Stage 2 Endpoints |

Primary

| | |
|---|---|
| To compare the efficacy of brazikumab with | Co-primary: Endoscopic response at Week 52 |
| that of Humira ® to achieve endoscopic | Co-primary: Clinical remission at Week 52 |
| response and clinical remission at Week 52 in | |
| participants who are BM+ | |

Secondary

| | |
|---|---|
| To compare the efficacy of brazikumab with | Key Secondary: Endoscopic response at both |
| that of Humira ® to achieve sustained | Week 12 and Week 52 |
| endoscopic response and clinical remission at | Key Secondary: Clinical remission at both |
| both Week 12 and Week 52 in participants | Week 12 and Week 52 |
| who are BM+ | |
| To compare the efficacy of brazikumab with | Key Secondary: Endoscopic remission at |
| that of Humira ® in achieving endoscopic | Week 52 |
| remission and clinical remission at Week 52 in | Key Secondary: Clinical remission at Week 52 |
| participants who are BM+ | |
| To compare the efficacy of brazikumab with | Key Secondary: CS-free endoscopic remission |
| that of Humira ® to achieve CS-free | at Week 52 |
| endoscopic remission and clinical remission at | Key Secondary: CS-free clinical remission at |
| Week 52 in participants who are BM+ | Week 52 |
| To compare the efficacy of brazikumab with that | CS-free endoscopic remission at Week 52 for |
| of Humira ® to achieve CS-free endoscopic | participants taking CS at Baseline |
| remission and clinical remission at Week 52 in | CS-free clinical remission at Week 52 for |
| participants taking CS at Baseline and BM+ | participants taking CS at Baseline |
| To compare the efficacy of brazikumab with | Endoscopic response at Week 12 |
| that of Humira ® to achieve endoscopic | Clinical remission at Week 12 |
| response and clinical remission at Week 12 in | |
| participants who are BM+ | |
| To compare the efficacy of brazikumab with | Endoscopic response at Week 12 and |
| that of Humira ® to achieve endoscopic | endoscopic remission at Week 52 |
| response at Week 12 and endoscopic | Clinical remission at both Week 12 and |
| remission at Week 52 and clinical remission at | Week 52 |
| both Week 12 and Week 52 in participants | |
| who are BM+ | |
| To compare the efficacy of brazikumab with that | Endoscopic response at Week 52 |
| of Humira ® to achieve CS-free endoscopic | Clinical remission at Week 52 |
| response and clinical remission at Week 52 in | |
| participants who are BM+ | |
| To evaluate PK and immunogenicity of | Population PK model of serum concentrations |
| brazikumab in participants who are BM+ | of brazikumab and analysis for serum |
| | anti-brazikumab antibodies |
| To characterize the exposure-response | Exposure-response model linking primary |
| relationships of brazikumab in participants who are | endpoints to metrics of model-predicted |
| BM+ | individual brazikumab exposures |
| To evaluate the safety and tolerability of | AEs, clinical laboratory values, vital signs, |
| brazikumab in participants who are BM+ | physical exams, ECGs |

Additional

| | |
|---|---|
| To compare the efficacy of brazikumab with | CS-free endoscopic response at Week 12 and |
| that of Humira ® to achieve CS-free | endoscopic remission at Week 52 |
| endoscopic response at Week 12 and CS-free | CS-free clinical remission at both Week 12 and |
| endoscopic remission at Week 52 and CS-free | Week 52 |
| clinical remission at both Week 12 and Week | |
| 52 in participants who are BM+ | |
| To evaluate the impact of brazikumab on the signs | Change from Baseline at Week 12 and Week 52 |
| and symptoms of CD in participants who are BM+ | in signs and symptom scores (eg, LSF, AP, urgency, |
| | fatigue) derived from the BSFS, NRS, CD-PRO, |
| | PGIS-CD, PIS-AP, PII-LBMF, PGIC-CD, and FACIT-F |
| To evaluate the impact of brazikumab on HRQoL | Change from Baseline at Week 12 and at |
| in participants who are BM+ | Week 52 in IBDQ, SF-36, and EQ-5D-5L |
| To explore changes in signs and symptoms and | Exploratory analysis of subscale scores from |
| HRQoL using a range of measures in participants | BSFS, CD-PRO, IBDQ, and Allergan- |
| who are BM+ | developed items |

Intervention Groups and Study Duration:

Stage 1

Subjects are divided into the following Treatment Groups. (1) Brazikumab high dose: intravenous (IV) brazikumab 1440 mg on Days 1, 29, and 57, followed by subcutaneous (SC) brazikumab 240 mg on Day 85 and every 4 weeks through Week 48; (2) Brazikumab low dose: IV brazikumab 720 mg on Days 1, 29, and 57, followed by SC brazikumab 240 mg on Day 85 and every 4 weeks through Week 48; (3) Humira® : SC Humira® 160 mg on Day 1, 80 mg on Day 15, and 40 mg beginning on Day 29 and every 2 weeks through Week 50; and (4) Placebo: IV placebo on Days 1, 29, and 57, followed by SC placebo on Day 85 and every 2 weeks through Week 50. Study duration is up to 66 weeks, consisting of a 4-week screening period, a 52-week treatment period, and an 18-week post-last brazikumab/brazikumab placebo dose safety follow-up period.

Stage 2

Stage 2 Screening will be initiated upon completion of Stage 1, Week 12 analysis results. Subjects will be divided into the following treatment groups: (1) Brazikumab high dose: IV brazikumab 1440 mg on Days 1, 29, and 57, followed by SC brazikumab 240 mg on Day 85 and every 4 weeks through Week 48; (2) Brazikumab low dose: IV brazikumab 720 mg on Days 1, 29, and 57, followed by SC brazikumab 240 mg on Day 85 and every 4 weeks through Week 48; and (3) Humira® : SC Humira® 160 mg on Day 1, 80 mg on Day 15, and 40 mg beginning on Day 29 and every 2 weeks through Week 50. The Study Duration is up to 66 weeks, consisting of a 4-week screening period, a 52-week treatment period, and an 18-week post-last brazikumab/brazikumab placebo dose safety follow-up period.

Approximately 2000 participants will be screened such that approximately 450 participants are randomized to 1 of the 4 treatment groups in Stage 1 and approximately 690 participants, stratified 2:1 by BM+/BM− status, are randomized to 1 of the 3 treatment groups in Stage 2.

Administration of brazikumab in BM+ participants will result in reduced intestinal inflammation, which will translate into an improved endoscopic response and clinical remission rate (as measured by the SES-CD and CDAI subscores of LSF and AP) compared with placebo (Stage 1) and with Humira® (Stage 2) in participants with moderately to severely active CD.

The current study is designed to combine both initial treatment (induction) and maintenance phases into a single study, in a ‘treat-straight-through’ approach. Using this design, participants are randomized to receive induction therapy with study intervention or active control (or placebo in Stage 1 only) and are then treated straight through for the remainder of the study, which includes both an assessment of endoscopic response and clinical remission at Week 12 and an assessment of sustained endoscopic response and clinical remission in participants who were in response and/or remission at both Week 12 and Week 52. The major advantage of this naturalistic design is that it allows evaluation of both induction and maintenance treatment in a single study and avoids some of the complexities noted above that are associated with a traditional re-randomization maintenance design. Also, the consolidation of the benefits of initial treatment can be evaluated with continued treatment, especially for those participants who have responded to the initial treatment but did not meet the endoscopic response or clinical remission criteria at Week 12 but could be converted to a responder/remitter with continued treatment. This naturalistic design also mimics clinical practice as patients would continue to be treated along a continuum and not have their treatment truncated into an artificially selected timepoint. Furthermore, preserving the initial randomization assignment to treatment would ensure that long-term maintenance treatment was not biased in favor of participants that achieved remission during the Induction Period because those who achieved remission with their treatment would still be on the same treatment in the maintenance phase, without any influence of withdrawal or discontinuation of the treatment. Additionally, those who responded to placebo (in Stage 1 only) during the induction phase would still be on placebo in the maintenance phase, without any influence of discontinuation of placebo.

The no-adverse-effect level of 300 mg/kg for brazikumab was established in cynomolgus monkeys in 3 studies in which brazikumab was administered IV weekly for up to 14 weeks and SC weekly up to 6 months. At this dose, no toxicologically significant effects were observed.

Table 3 presents the margins of exposure calculated for the doses proposed in this study.

TABLE 3
Exposure Margins Supporting Planned Doses

| | Clinical 700 mg IV dose^{a} | | Clinical 1400 mg IV dose^{b} | | Clinical 2100 mg IV dose^{c} | |
|---|---|---|---|---|---|---|
| | AUC_{0-28 days} | Cₘₐₓ | AUC_{0-28 days} | Cₘₐₓ | AUC_{0-28 days} | Cₘₐₓ |
| Margin of Exposure | 54.2 | 47.4 | 27.1 | 23.7 | 22.2 | 16.2 |

^{a}Phase 1b Study 20090519
^{b}Estimated values based on exposure in Phase 1b Study 20090519
^{c}Phase 1 Study 3150-101-008

Humira® is used as an active control for Stage 1 to provide internal evidence of assay sensitivity and as an active comparator for Stage 2. Participants in the placebo-and active-comparator groups will undergo the same study assessments as the brazikumab-treated participants.

### Study Population

Inclusion and Exclusion Criteria are the same for both Stage 1 and Stage 2; however, participants enrolled in Stage 1 will not be permitted to enroll in Stage 2.

### A. Inclusion Criteria

Participants are eligible to be included in the study only if all of the following criteria apply: (1) Aged 16 to 80 years, inclusive, or minimum age of adult consent according to local regulations, at Screening. For participants less than 18 years of age, the participant must weigh at least 40 kg at Screening; (2) Diagnosis of ileal, ileocolonic, or colonic CD with an onset of symptoms for a minimum of 3 months prior to Screening as determined by the investigator based on clinical history, exclusion of other etiologies including infectious causes, and characteristic endoscopic and/or histologic findings; (3) Moderately to severely active CD, defined by the following (3a and 3b must be met): (a) CDAI LSF and AP scores are obtained during Screening on an e-diary. The calculation of LSF and AP for eligibility is based on the participant's evening diary data that will be collected daily during Screening. Diary data from the days for familiarization with the device (2 days), bowel preparation, and the endoscopy procedure will not be used for eligibility calculations. The eligibility calculation is based on a 7-consecutive-day period that does not include bowel preparation and endoscopy. Within the 7-consecutive-day period, participants are to have at least 4 days of evening diary entries. The following criteria must also be met: (i) Average daily CDAI LSF score ≥5 OR, (ii) Average daily CDAI AP score ≥2; (iii) Evidence of active intestinal mucosal inflammation, as demonstrated on video-recorded ileocolonoscopy performed within 35 days prior to Day 1 and scored by a blinded central reader with agreement on the following findings: (1) SES-CD score of at least 6. A narrowing that cannot be passed is exclusionary. The SES-CD score is calculated based on segments that can be evaluated by the endoscopist, OR (iv) For isolated ileal disease, SES-CD score of at least 4. All efforts are made to complete the ileocolonoscopy no less than 3 business days prior to IWRS randomization to allow for the evaluation of the endoscopic subscore by the central reader.

(4) Participant had an inadequate response or intolerance to intervention with oral aminosalicylates, oral corticosteroid (CS), azathioprine, methotrexate, or 6-mercaptopurine, or demonstrated CS dependence for the treatment of CD. To fulfill this criterion the participant must meet at least 1 of the following: (a) Had an inadequate response to one of these agents, i.e., defined as persistent signs and/or symptoms of active CD judged by the investigator's overall clinical assessment of the participant's history taking into consideration a lack of clinical improvement or inability to maintain previously achieved clinical improvement despite treatment with medication(s) used according to the local label and generally considered to be safe and effective in treating CD; (b) Was intolerant to one of these agents, defined as the inability to continue treatment due to adverse effects, regardless of treatment dose; (c) Has CS dependence, defined as the daily or regularly scheduled use of CS to manage CD signs/symptoms and inability to discontinue CS use without the prompt return of CD signs/symptoms.

(5) Where applicable, participants taking any of the following medications must be at a stable dose as defined: (a) 5-aminosalicylates must be at a stable dose for 2 weeks prior to Baseline (Visit 2)′ (b) Oral prednisone (or equivalent) up to 25 mg/day or equivalent, must be at a stable dose for 2 weeks prior to the eligible colonoscopy and kept stable until the Week 12 (Visit 9) assessment; (c) Budesonide up to 9 mg/day, must be at a stable dose for 2 weeks prior to Screening colonoscopy and kept stable until the Week 12 (Visit 9) assessment; (d) Immunomodulators (specifically azathioprine, 6-mercaptopurine, and methotrexate): participant must have been on treatment for a minimum of 8 weeks and must be kept at stable doses (except for cases of toxicity when the dose may be lowered) for 2 weeks prior to Baseline (Visit 2); (e) Oral antibiotics for the treatment of CD must be at a stable dose at Baseline (Visit 2). This criterion does not apply to antibiotics used for the treatment of active infection; (f) Probiotics (e.g., Culturelle® and Saccharomyces boulardii) must be at a stable dose at Baseline (Visit 2).

(6) Meets the following TB criteria, and TB worksheet has been completed: (a) Participant has no known history of active TB; (b) Participant has no known history of latent TB without completion of an appropriate course of intervention or is presently taking appropriate ongoing prophylactic intervention; (c) Meets one of the following acceptable TB test results: (i) Negative QFT-TB obtained from central laboratory within 4 weeks prior to randomization, OR (ii) For a positive QFT-TB test obtained during Screening from the central laboratory, active TB must be ruled out. For newly positive QFT-TB results, treatment for latent TB must be initiated prior to the first dose of study intervention, and participant agrees to complete the full duration of prophylaxis. If not newly positive, there is to be documentation that a full course of prophylaxis for latent TB was completed or will be initiated and completed. No evidence of active TB on chest x-ray within 8 weeks prior to Screening or during Screening. Participants in countries with high multidrug resistant TB burden with a new diagnosis of latent TB during Screening will be excluded, OR (iii) Indeterminate QFT-TB test (confirmed as indeterminate on retest during Screening) obtained during the Screening period from the central laboratory with ongoing QFT-TB testing. Participants with an indeterminate QFT-TB test can continue with Screening if they have all of the following: (1) No symptoms per TB worksheet provided by the sponsor; (2) No known recent exposure to a case of active TB; (3) No evidence of active TB on chest x-ray within 8 weeks prior to Screening or during Screening; or (iv) A negative tuberculin skin test is required if the QFT-TB test is not approved/registered in that country. QFT-TB test must also be performed and i, ii, or iii above must be met. (d) Participants with a history of using anti-TNFα agents for a treatment course of one year or longer who have discontinued an anti-TNFα agent within 6 months prior to Screening must obtain a chest x-ray showing no evidence of active TB within 8 weeks prior to Screening or during Screening.

(7) Females of childbearing potential who are sexually active with a nonsterilized male partner must use two acceptable methods of contraception, one of which must be a highly effective method, and must agree to continue using such precautions for 18 weeks after the last dose of investigational product; cessation of contraception after this point is to be discussed with a responsible physician. Periodic abstinence, the rhythm method, and the withdrawal method are not acceptable methods of contraception. All female participants of childbearing potential must have a negative serum β-human chorionic gonadotropin level by the central laboratory prior to randomization. (a) Females of childbearing potential are defined as those who are not surgically sterile (i.e., bilateral salpingectomy, bilateral oophorectomy, or complete hysterectomy) or those who are not postmenopausal defined as 12 months with no menses without an alternative medical cause; (b) Participants must not donate or bank egg cells for fertilization purposes from Screening up to 18 weeks after the last dose of study intervention.

(8) Female participants who are post-menopausal and of non-childbearing potential must have an elevated FSH at or above the range for post-menopausal women by the central laboratory during Screening. Post-menopausal status is defined as an absence of menses for at least 1 year from the time of the last menses.

(9) Nonsterilized males who are sexually active with a female partner of childbearing potential must comply with the methods of contraception described herein and for 18 weeks after the last dose of investigational product and must not donate or bank sperm for fertilization purpose for the same time period.

(10) Ability to provide written informed consent prior to any study procedures (Appendix 10.1).

(11) Willingness and ability to attend all study visits, comply with the study procedures, read and write in order to complete questionnaires, and be able to complete the study period.

### B. Exclusion Criteria

Participants are excluded from the study if any of the following criteria apply: (1) Participant has previously received Humira® and was intolerant to treatment or had met the criteria for primary or secondary non-response to treatment: (a) Primary non-response: Signs and symptoms of persistently active disease despite a history of at least 1 induction regimen of Humira® per the local label consisting of at least 2 doses at least 2 weeks apart; (b) Secondary non-response: Recurrence of symptoms of persistently active disease during scheduled maintenance dosing of Humira® per the local label following prior clinical benefit; (c) Intolerance: AE associated with discontinuation of Humira® therapy, including, but not limited to, hypersensitivity, infusion-related reaction, infection, or congestive heart failure. (2) Participant is unable or unwilling to have endoscopic procedures performed during the study. (3) History or current diagnosis of ulcerative colitis, indeterminate colitis, microscopic colitis, ischemic colitis, colonic mucosal dysplasia, primary sclerosing cholangitis, or untreated bile acid malabsorption. (4) History of toxic megacolon within 3 months prior to Baseline (Visit 2). (5) Any intra-abdominal surgery, bowel resection, diversion, placement of ostomy or stoma within 3 months prior to Screening. Participants with a draining stoma are excluded. (6) Participant has an enterocutaneous or enterovesicular fistula. Participants with active fistulas may be considered if there is no anticipation for surgery and there is no evidence of active infection (eg, abscess) after further discussion with the study medical monitor. (7) Bowel perforation during the 6 months prior to Screening or evidence of obstruction within 3 months of Screening. (8) Complications of CD including short bowel syndrome, strictures/stenoses with obstruction or pre-stenotic dilation, or conditions where surgery may be anticipated within 6 months, or other conditions that may confound efficacy evaluations for the study. (9) Participant has any non-passable colonic stenosis/narrowing identified during the qualifying ileocolonoscopy (successful endoscope passage to the caecum with inability to enter the endoscope into the ileum is not covered under this exclusion criterion, and does not require exclusion). (10) Ongoing nutritional dependency for total parenteral nutrition or an elemental diet at Screening. (11) Participant has any of the following related to infections: (a) Evidence of a recent (within 6 months of Baseline [Visit 2]) systemic fungal infection, requiring inpatient hospitalization, and/or antifungal treatment. Participants treated for localized fungal infections (e.g., oral, vaginal, or skin candidiasis, onychomycosis) are not excluded; (b) Any infection requiring hospitalization or treatment with IV anti-infectives (including anti-viral treatment) within four weeks of Screening; (c) Cytomegalovirus or Epstein-Barr virus infection that has not completely resolved within 8 weeks prior to Screening; (d) Clinically significant chronic infection (eg, osteomyelitis) that has not resolved within 8 weeks of Screening; (e) Non-serious infection requiring oral anti-infectives within 2 weeks prior to randomization must be further discussed with the study medical monitor. Chronic suppressive antiviral treatment for herpes simplex virus in the absence of active lesions or uncomplicated urinary tract infections are not considered exclusionary; (f) Participant has clinical evidence of or suspected to have an abscess during Screening. Cutaneous and perianal/perirectal abscesses are not exclusionary if drained and adequately treated at least 3 weeks prior to Screening; (g) Diagnosis of peritonitis or receiving treatment for peritonitis within 8 weeks prior to Screening; or (h) Participant has any underlying condition that predisposes participant to infections. (12) Previous allogenic bone marrow transplant or history of organ or cell-based transplantation (eg, islet cell transplantation or autologous stem cell transplantation) with the exception of corneal transplant. (13) Chronic hepatitis B or C infection, TB, or C. difficile-positive at Screening (Visit 1). (14) Known history of primary immunodeficiency, splenectomy, or any underlying condition that predisposes the subject to infection, including HIV infection. Participants with positive results of HIV testing by the central laboratory will be excluded. (15) Prior history of or current diagnosis of a demyelinating disorder. (16) Participant has received the following treatment: (a) Infliximab: within 8 weeks prior to Baseline (Visit 2); (b) Humira® , certolizumab pegol, or golimumab: within 10 weeks prior to Baseline (Visit 2); (c) Vedolizumab within 18 weeks prior to Baseline (Visit 2); (d) Ustekinumab within 14 weeks prior to Baseline (Visit 2); or (e) Other prohibited medication, biologic or small molecule treatment within 5 half-lives prior to Baseline (Visit 2). (17) Except for ustekinumab, prior exposure to any biologic agent targeting IL-12 or IL-23 (e.g., risankizumab, briakinumab, mirikizumab, guselkumab, tildrakizumab,or brazikumab). (18) Participants who received cyclosporine, mycophenolate mofetil, sirolimus (rapamycin), thalidomide, tacrolimus (FK-506), or tofacitinib within 2 weeks prior to Screening. (19) Known history of allergy to the study intervention formulation or any of its excipients or components of the delivery device, or to any other biologic therapy. (20) Participants received IV or intramuscular steroids within two weeks prior to Screening. (21) Participant received topical (rectal) aminosalicylic acid (eg, mesalamine) or topical (rectal) steroids within two weeks prior to Baseline (Visit 2). (22) Participant received a Bacille Calmette-Guérin vaccination within 12 months of Baseline (Visit 2) or any other live vaccine less than four weeks prior to Baseline (Visit 2) or is planning to receive any such vaccine over the course of the study. (23) Participant has known history of drug (including opiates) or alcohol abuse within one year of Screening. Participants who use marijuana for medicinal purposes, including treatment of symptoms associated with CD and improving quality of life, will be permitted in the study. Marijuana use is to be documented as a concomitant medication. Participants who abuse marijuana (i.e., interferes with aspects of the participant's life) as judged by the investigator are excluded. (24) History of cancer with the following exceptions: (a) A history of basal cell carcinoma and/or squamous cell carcinoma of the skin, with apparent successful curative therapy, would not be exclusionary within the following time periods: (i) Greater than 12 months prior to screening, if the participant has previously been, or currently is, on thiopurine treatment; (ii) Greater than 3 months prior to screening, if the participant has no prior history of, or current, thiopurine use; (b) Carcinoma in situ of the cervix, with apparent successful curative therapy, greater than 12 months prior to screening. If there is evidence of intestinal epithelial dysplasia on endoscopy, and confirmed on biopsy, the participant must be excluded. (25) Clinically significant cardiovascular conditions including recent myocardial infarction, unstable angina, stroke, transient ischemic attack, decompensated heart failure requiring hospitalization, or Class III/IV heart failure within 6 months of Screening. (26) Prolonged QTcF interval (determined on central ECG), or conditions leading to additional risk for QT prolongation (eg, congenital long-QT syndrome). Participants with electrolyte abnormalities such as hypokalemia and hypomagnesemia that would increase the risk of QT prolongation are to be corrected prior to randomization; the ECG for these participants may be repeated after electrolyte correction for determination of eligibility if needed. (27) Clinically significant kidney disease including but not limited to: (a) Acute kidney injury within 6 weeks of Screening. Corrected pre-renal azotemia with serum creatinine at the participant's Baseline value during Screening would not be excluded; (b) Chronic kidney disease with an estimated glomerular filtration rate of less than 30 ml/min calculated by MDRD equation or Schwartz equation, as applicable, by the central laboratory at Screening are excluded. (28) Abnormal laboratory results at Screening (Screening window may be extended to obtain screening test results after discussion with medical monitor): (a) Liver tests: either AST, ALT, or alkaline phosphatase >2.0×ULN or total bilirubin >1.5×ULN (except for subjects with Gilbert Syndrome); (b) Neutrophil count <1×10₃/μl (or <1.0 GI/L); (c) Hemoglobin <8 g/dL; (d) Platelet count <100×103/μl (or <100 GI/L); (e) Evidence of acute or chronic hepatitis B or C infection on central laboratory serology; (f) Positive central laboratory result for HIV; (g) C. difficile-positive stool testing by central laboratory; (h) Participant has any other abnormal laboratory results at Screening, which, in the opinion of the investigator, will prevent the participant from completing the study or will interfere with the interpretation of the study results. (29) Participant has any kind of disorder that, in the opinion of the investigator, may compromise the ability of the participant to give written informed consent and/or to comply with all required study procedures. (30) Participant is currently enrolled in another investigational device or drug study, or is within than 35 days or five half-lives, whichever is longer, since ending another investigational device or drug study, or receiving other investigational agent(s). In the event that a participant has received an investigational agent, the elimination half-life of which is not known, then the last dose must have been received at least 6 months prior to Baseline (Visit 2). (31) Transfusion of blood, plasma, or platelets within the 30 days prior to Screening. (32) Participant is pregnant, breastfeeding, or plans to become pregnant during the study. (33) Employees of the clinical study site or any other individuals involved with the conduct of the study, or immediate family members of such individuals.

### Rationale for Inclusion and Exclusion Criteria

Participants in this study will be 16 to 80 years of age, inclusive, with moderately to severely active CD who, as determined by the investigator, have failed or are intolerant to conventional therapy. This includes participants who have not received a biologic agent (biologic naive) or have received a biologic agent (e.g., anti-TNFα or anti-integrin) at a dose approved for the treatment of CD and did not respond initially (i.e., primary non-response), or responded initially but then lost response with continued therapy (i.e., secondary non-response), or were intolerant to the medication. This also includes participants who have previously received a biologic agent with a successful response without subsequent treatment failure. However, since Humira® is used as an active comparator, participants who have failed (met the criteria for primary or secondary nonresponse to treatment) or are intolerant to prior treatment with Humira®will be excluded.

In the Phase 2a study (CD-IA-MEDI2070-1147), brazikumab demonstrated efficacy, without an identified safety risk, in a population of 18 to 65 years of age with moderate to severe, active CD. This study seeks to confirm and expand upon those observations, and to extend them into a population of 16 to 80 years of age. Most currently available treatments for moderately to severely active CD, including glucocorticosteroids, immunomodulators, and anti-TNFα agents are associated with significant adverse effects. The mechanism of action of brazikumab, and the results of the Phase 2a study in subjects with CD, indicates that brazikumab has the potential to offer effective treatment with a reduced risk of adverse effects.

Despite the availability of current treatments, there is still a need for novel therapies for the treatment of CD due to the evidence that not all patients will respond or maintain their response to the available treatment options. As such, a considerable proportion of patients with moderately to severely active CD are unresponsive to both conventional therapy and current biological therapy, and considerable unmet medical need remains among these patients for safe and effective long-term therapy.

### Study Intervention

Study intervention is defined as any investigational treatment(s), marketed product(s), placebo, or medical device(s) intended to be administered to a study participant according to the study protocol.

Participants who satisfy all inclusion and exclusion criteria will receive study intervention, defined as any investigational treatment or placebo intended to be administered to a study participant according to the study protocol. Table 4 presents details regarding study intervention and administration.

TABLE 4
Stage 1 and Stage 2 Study Interventions

| Study | | | | Placebo |
|---|---|---|---|---|
| intervention Name | Brazikumab IV | Brazikumab SC | Humira ® | (Stage 1 only) |
| Route of | IV infusion | SC injection | SC injection | SC injection and |
| Administration | | | | IV infusion |
| | 720 | 240 | 160 | 0 |
| | | | (Day 1) | |
| Dose | | | 80 | |
| Strength, mg | | | (Day 15) | |
| | 1440 | | 40 | |
| | | | (Day 29 and every | |
| | | | 2 weeks through | |
| | | | Week 50) | |
| Dosing | Administer | Administer on | Administer on | IV: Administer |
| Instructions^{a} | 60-minute infusion | Day 85 and every | Day 1 and every | 60-minute |
| | on Day 1, Day 29, | 4 weeks through | 2 weeks through | infusion on |
| | Day 57 | Week 48 | Week 50 | Day 1, Day 29, |
| | | | | Day 57 |
| | | | | SC: Administer |
| | | | | every 2 weeks |
| | | | | through Week 50 |
| Packaging and | Study | Study | Study | SC dosing: |
| Labeling | intervention | intervention | intervention | Brazikumab placebo |
| | will be provided in | will be provided in | will be provided in | will be provided in |
| | kits. Each kit will | kits. Each kit will | kits. Each kit will | kits. Each kit will |
| | be labeled as | be labeled as | be labeled as | be labeled as |
| | required per | required per | required per | required per |
| | country | country | country | country |
| | requirement. | requirement. | requirement. | requirement. |
| | | | | IV dosing: Study |
| | | | | intervention will |
| | | | | not be provided. |

^{a}For IV infusions, contents of vials will be added to an IV bag of dextrose solution to a total volume of 100 mL then administered by IV infusion.

### A. Double-Dummy Dosing Regimen

In both stages of this protocol, brazikumab is administered as a 60-minute 100 mL IV infusion for the first 3 doses and SC injection for all subsequent doses; all Humira®doses are administered as SC injections. Therefore, because the preparations of brazikumab and Humira® are distinct in appearance and volume, special precautions need to be taken to ensure the double-blind nature of the study. The double-dummy technique will be used to maintain the blind when administering the treatments because the brazikumab and Humira® treatments cannot be made identical. All participants will be administered the same number and type (e.g., IV and/or SC) of treatments throughout the study regardless of treatment group assignment. For example, during the Induction Period, an IV infusion and SC injections will be administered to each participant for induction doses on Study Days 1, 29, and 57 (Visits 2, 5, and 7); only SC injections will be administered on Study Days 15, 43, and 71 (Visits 4, 6, and 8).

i. Intravenous Administration

All participants will receive one IV infusion of study intervention (brazikumab, sham placebo) on Days 1, 29, and 57 (Visits 2, 5, and 7) of the Induction Period. An experienced and qualified staff member will place the IV access.

The IV study intervention (brazikumab or sham placebo) will be delivered in 5% w/v dextrose in water in a volume of 100 mL over a minimum of 60 minutes using an infusion pump. Before and after the IV infusion, the IV access will be flushed with 30 mL of 5% w/v dextrose in water.

Vital signs (BP, temperature, pulse rate, and respiration rate) will be obtained before IV study intervention administration at all treatment visits. In addition, participants will be monitored for changes in vital signs and/or new symptoms approximately every 15 minutes during IV administration, immediately after completion of infusion, and at approximately every 30 minutes for a minimum of one hour post-infusion or until stable, whichever is later. The first and last vital signs are to be recorded on the eCRF. Participants will be discharged from the site when they are deemed clinically stable by the investigator, a minimum of one hour after completion of IV administration for the initial two infusions (Visits 2 and 5). The monitoring time after the third infusion (Visit 7) may be reduced to a minimum of 30 minutes at the discretion of the investigator.

Infusion reactions have been reported with the administration of IV monoclonal antibodies. As with any antibody, allergic reactions to dose administration are possible. Appropriate drugs, such as epinephrine, antihistamines, CS, and medical equipment to treat anaphylactic reactions must be immediately available at study sites, or procedures for emergency treatment must be in place. Study personnel must be trained to recognize and take appropriate action for emergency measures according to local guidelines. Any infusion reaction and/or hypersensitivity reaction is to be reported as an AESI (see Sections on Infusion Reactions and Injection-site Reactions and Hypersensitivity Reactions).

ii. Subcutaneous Administration

Brazikumab, Humira®, or sham placebo will be administered to all participants during the Induction and Maintenance Periods by SC injection. Each SC dose will be administered to the participant's anterior abdominal wall by an unblinded, experienced and qualified staff member. The brazikumab, Humira®, or sham placebo dose will be administered as a single or multiple SC injections according to the double-dummy dosing administration table. Injections will be on alternating (left or right) sites on the participant's anterior abdominal wall over no more than 10 minutes total time for all SC injections and at a distance of at least two cm apart.

Vital signs (BP, temperature, pulse rate, and respiration rate) will be obtained before and immediately after SC study intervention administration during treatment visits. In addition, for Visit 9 and Visit 11 (first two SC brazikumab doses) participants will be monitored for changes in vital signs and/or new symptoms approximately every 30 minutes for a minimum of one hour post-injection or until stable, whichever is longer. For the third and subsequent SC doses of brazikumab or placebo, participants will be monitored for a minimum of 30 minutes or until stable, whichever is longer. The first and last vital signs (pre- and post-dose) are to be recorded on the eCRF. Discharge from the site will be determined by the investigator. Any injection site reaction is to be reported as a TEAE.

Study intervention accountability will be performed for each site during the course of the study, and all study intervention must be accounted for. All unused study intervention must be stored securely with access limited to the unblinded personnel and returned to the sponsor or designee once expired or at the termination of the study. Empty vials may be destroyed onsite by the unblinded personnel after accounted for by the unblinded monitor.

### Efficacy Assessments

### A. Primary Efficacy Assessments

i. Ileocolonoscopy

Ileocolonoscopy procedures will be recorded using a video capture kit as supplied by the central reading facility. All video recordings will be labeled with segment names by the central reader vendor to produce a complete ileocolonoscopy video visualized up to the terminal ileum. A complete endoscopic video may not include the terminal ileum if it cannot be visualized. The video clips will be read centrally for mucosal lesions and endoscopic severity based on the SES-CD score by independent gastroenterologists experienced in IBD who are blinded to the subject's clinical activity and treatment allocation. The worst affected area of each segment is to be assessed for the SES-CD score calculation.

In all cases, video recordings are to be performed prior to biopsy. Technical instructions for making the video recording will be provided separately (these instructions will include how to capture the depth of insertion and how to mark bowel segments during the recording).

The central readers are to promptly notify both the medical monitor and the investigator of the detection of any clinically significant bowel lesions that are not manifestations of CD.

Ileocolonoscopies will be performed: (a) Prior to Baseline (Visit 2) for eligibility assessment, (b) At the end of the Induction Period: Week 12 (Visit 9), (c) At the end of the Maintenance Period at Week 52 (Visit 29), and (d) At any early termination visits.

To ensure quality data and standardization, the same endoscopist for a participant is to be used throughout the study whenever possible. Ileocolonoscopies will be read at a centralized reading facility, with the central readers blinded to the participant's clinical activity and treatment allocation.

ii. Simple Endoscopic Score for Crohn's Disease

The SES-CD is a validated endoscopic activity score used to assess the status and change of mucosal lesions in patients with CD (Daperno 2004). The score assesses 4 variables in up to 5 segments to yield its final result (Table 5).

The 5 segments assessed are: (1) Rectum, defined as that portion distal to the rectosigmoid junction, (2) Left colon including the sigmoid colon, (3) Transverse colon defined as the segment between the hepatic and the splenic flexures, (4) Right colon including the ileocecal valve, cecum, and ascending colon to the hepatic flexure, and (5) Ileum.

TABLE 5
Simple Endoscopic Score for Crohn's Disease Values

| Variable | 0 | 1 | 2 | 3 |
|---|---|---|---|---|
| Size of ulcers | None | Aphthous | Large | Very |
| | | ulcers | ulcers | large ulcers |
| Ulcerated surface | None | <10% | 10-30% | >30% |
| Affected surface | Unaffected | <50% | 50-75% | >75% |
| | segment | | | |
| Presence of | None | Singl | Multiple, | Cannot |
| narrowings | | can be | can be | be |
| | | passed | passed | passed |

SES-CD for each of the 5 segments will be assessed during ileocolonoscopy prior to Baseline (Visit 2) for eligibility assessment, at the end of the Induction Period: Week 12 (Visit 9), at the end of the Maintenance Period at Week 52 (Visit 29), and at any early termination visits.

iii. Biopsy

Mucosal biopsies will be collected at each study endoscopy (prior to Visit 2, 9, 29, and/or Early Termination Visit). At least 2 biopsies per segment (total of 5 segments) are to be obtained, focusing on the areas of greatest inflammation or areas of ulceration within each segment. If no inflammation or ulceration is present, then random biopsies of the segment are to be obtained. The biopsies will be used to support assessments of changes over time. Histological indices that will be used for evaluation of the biopsies will be detailed in the SAP.

Detailed instructions for biopsy collection, kits for processing, handling, and shipping will be provided to the sites, to support the centralized testing for each of the various exploratory objectives. A central laboratory will be used to process and stain the biopsy specimens.

iv. Crohn's Disease Activity Index

The LSF and AP items from the CDAI will be used for the primary and secondary PRO assessments.

The CDAI is a composite index with weighted domains that quantifies the global disease severity in a single numerical score. The CDAI measures the severity of active disease using symptom scores that are monitored over the previous week and includes subject-reported symptoms, physician-assessed signs, and laboratory markers (Best 1976, Sands 2005). The CDAI score has historically been the gold standard for the assessment of efficacy in clinical trials in CD, however regulatory agencies such as FDA have recently indicated that they no longer consider it fit for purpose to support registration. However, the full CDAI will be assessed in this study as an exploratory endpoint to perform indirect treatment comparisons with other therapies.

Participant-reported components of the CDAI LSF and AP will be collected daily via electronic diary. The participant will be prompted by the daily diary every evening during the Screening and Induction Period. During the Maintenance Period, participants will be required to fill out the evening diary only 1 week out of every 4 weeks from Week 12 to Week 47. The rest of the time the diary will be unavailable for completion. Starting at Week 48, the evening diaries will be available every day.

### B. Secondary Efficacy Assessments

The SES-CD and CDAI (LSF and AP) will also be used for secondary assessments. Serum brazikumab concentrations for the conduct of population PK analysis and exposure-response model linking of primary endpoints to metrics of model-predicted individual brazikumab exposures are determined. Serum IL-22 concentrations are measured. Also, the safety and tolerability of brazikumab (AEs, clinical laboratory values, vital signs, physical examinations, and ECGs) are assessed.

### C. Additional Efficacy Assessments

i. Bowel Movement eDiary

Information will be captured by participants after each bowel movement, which is defined as a trip to the toilet when the participant passes stool, blood, or mucus. They will record the time of occurrence, bowel movement components (stool, blood, or mucus), presence or absence of urgency, and the stool consistency using the BSFS.

The BSFS classifies the form of stools into 7 types, each with an accompanying visual aid and text description. The BSFS will be used to measure the form of individual stools, with loose/liquid stools characterized by either Type 6 (fluffy stool pieces with ragged edges, a mushy stool) or Type 7 (all liquid) on the scale of Type 1 (hard lumps of stool) to Type 7 (all liquid).

ii. Evening eDiary

At the end of the day, during the evening diary, participants will be prompted to add any additional bowel movements that they did not enter in real-time.

Participants will also be instructed to enter CDAI LSF and AP items and NRS items (AP, fatigue, tiredness, weakness, lack of energy, joint pain). The NRS measures the severity of symptoms in the past 24 hours using an 11-point Likert scale (0-10).

The evening e-diary will be prompted every evening during the Screening and Induction Period. During the Maintenance Period, evening e-diaries will be entered every evening for 1 week out of 4 for Weeks 13 to 47. Starting at Week 48, participants will recommence daily entering of the bowel movement and evening diaries.

iii. Extended Evening eDiary

At Screening, Baseline, Week 12, and Week 52, an extended evening e-diary will also include a CDAI item asking about presence or absence of temperature higher than 100° F. (37.8° C.) and the CD-PRO daily recall modules.

The CD-PRO is a 38-item disease-specific instrument that is currently under development. The CD-PRO is designed to be self-administered and measures symptoms and impacts on patients' lives (Higgins 2013). The CD-PRO consists of 3 modules that use daily recall to record concepts related to bowel movements (2 items), symptom severity (12 items), and coping activities (6 to 7 items), and 2 modules that use weekly recall items to capture emotional impact (8 items) and impact on patients' daily life (9 items).

Thus, daily recall items will be collected on a daily basis via e-diary. The 1-week recall items will be assessed at visits. When possible, the CD-PRO is to be completed by the subject prior to any non-PRO assessments and before the subject receives any disease-status information or study intervention during that visit.

iv. Weekly eDiary

At the end of each week, a weekly diary will be available after the nightly diary. It will include PGIS-CD, PIS-AP, PII-LBMF, PGIC-CD, and FACIT-F.

The weekly diary will be prompted weekly during the Screening and Induction Period. During the Maintenance Period, subjects will be required to fill out the weekly diary once every 4 weeks, around the time of their clinic visit for Weeks 13 to 47. The rest of the time the diary will be unavailable for completion. Starting at Week 48, participants will recommence weekly diaries.

The PGIS-CD is a single item that assesses participants' perceptions of overall severity of CD symptoms for the last seven days, with response options ranging from “none” to “severe.”

The PIS-AP is a single item that assesses participants' perceptions of overall severity of AP for the last seven days, with response options ranging from “none” to “severe.”

The PII-LBMF is a single item that assesses participants' perceptions of the level of interference in activities of daily living due to loose bowel movement for the last 7 days with response options ranging from “never” to “always.”

The PGIC-CD is a single item that assesses participants' perceptions of overall change in their CD symptoms over the last 7 days. This item will be available starting on Week one.

The FACIT-F Scale (Version 4) is a 13-item instrument that measures fatigue and its impact on daily functions over a recall period of 7 days. Five of the items assess the experience of fatigue, and eight items assess the impact of fatigue. Items are scored on a 5-point Likert scale, yielding a score ranging from 0 to 52, with lower scores indicating greater fatigue. FACIT-F has been widely used in clinical trials and with participants with IBD (Tinsley 2011). FACIT-F is designed to be self-administered and can be completed in under five minutes.

v. Site Visit Instruments

During visits, sites will initiate an electronic data collection tool that will administer both participant-reported items and clinician-reported CDAI items. Participants will complete the IBDQ, EQ-5D-5L, SF-36, and CD-PRO weekly recall modules at the visits specified in the SoA.

The IBDQ is a disease-specific PRO instrument that measures HRQoL in patients with IBD (Guyatt 1989). The IBDQ covers the following dimensions: bowel symptoms (10 items), systemic symptoms (five items), emotional function (12 items), and social function (five items). Items are scored on a seven-point Likert scale, yielding a global score in the range 32 to 224 (with higher scores indicating better quality of life). The IBDQ has been frequently used in drug approval applications to assess treatment efficacy in IBD. The IBDQ has been designed to be self-administered and completed in five minutes.

The EQ-5D-5L is a standardized instrument used to measure self-reports of health status and functioning, consisting of five elements: mobility, self-care, usual activities, pain/discomfort, and anxiety/depression. Empirically derived weights can be applied to an individual's responses to the EQ-5D-5L descriptive system to generate an index measuring the value to society of his or her current health. In addition, the EQ-5D-5L includes a VAS that allows respondents to rate their own current health on a 101-point scale ranging from “best imaginable” to “worst imaginable” health.

The SF-36 is a standardized instrument used to measure self-reports of health status and functional well-being, consisting of 8 domains: physical functioning, role physical, bodily pain, general health, vitality, social functioning, role emotional, and mental health. Empirically derived weights can be applied to an individual's responses to the SF-36 descriptive system to generate an index measuring the value to society of his or her current health.

### Biomarkers and Other Assessments

Blood and stool samples will be collected and analyzed to evaluate protein, nucleic acid, and cellular BM that relate to brazikumab intervention according to the SoA (see Table 9). All BM analyses will be conducted to generate hypotheses associated with the mechanisms of action of brazikumab, identify subsets of participants responsive to brazikumab, and to characterize a gene signature. Specific procedures for sample collection, processing, storage, and shipment can be found in a separate Laboratory Manual provided to the sites. All BM results will be summarized in a separate report at the conclusion of the study; sites will remain blinded to these results.

Whole blood samples will be collected in PAXgene RNA and DNA tubes for total RNA and DNA sample preparations. RNA may be used in the analyses of transcript expression using Thermo Fisher Clarion D array and stored for future analyses. DNA will be used in specific mutation analysis or whole genome sequencing as needed. PAXgene sample collections may not be obtained within 120 days of a nonleukocyte-depleted whole blood transfusion.

Venous blood samples will be collected for measurement of IL-22 serum concentration as specified in the SoA. Venous blood samples will be collected for measurement of K2EDTA plasma LCN2 concentration. Instructions for the collection and handling of biological samples will be provided by the sponsor. The actual date and time (24-hour clock time) of each sample will be recorded.

A separate set of blood serum/plasma samples will be collected for analysis of circulating soluble factors in relation to inflammatory cell activities. Factors to be analyzed may include, but are not limited to: IFN-γ, IL-6, IL-8, IL-10, IL-12, IL-17A, IL-2, IL-23, IL-22 binding protein, and TNFα. Protein analytes will be assessed by validated immunoassays as needed.

The BM analysis is exploratory and will be described in a separate report.

TABLE 6
Abbreviations and Trademarks

| Term/Abbreviation | Definition |
|---|---|
| ADA | anti-drug antibodies |
| AE | adverse event |
| AESI | adverse event of special interest |
| ALT | alanine aminotransferase |
| AP | abdominal pain |
| AST | aspartate aminotransferase |
| AUC | area under the serum concentration |
| | time-curve |
| BM | biomarker |
| BM− | serum IL-22 concentrations below a |
| | pre-established cutoff |
| BM+ | serum IL-22 concentrations at or above a |
| | pre-established cutoff |
| BP | blood pressure |
| BSFS | Bristol Stool Form Scale |
| CD | Crohn's disease |
| CDAI | Crohn's Disease Activity Index |
| CDISC | Clinical Data Interchange Standards Consortium |
| CD-PRO | Crohn's Disease Patient-Reported Outcome Scale |
| clinical | average daily LSF subscore of ≤3 as assessed |
| remission | on the CDAI LSF item AND |
| | average daily AP subscore of ≤1 as assessed |
| | on the CDAI AP item |
| clinical | minimum 25% reduction in LSF subscore or AP |
| response | subscore from Baseline |
| Cₘₐₓ | maximum concentration |
| CS | corticosteroids |
| CS-free | free of corticosteroids for the last 12 weeks |
| | before the assessment |
| DNA | deoxyribonucleic acid |
| ECG | electrocardiogram |
| eCRF | electronic case report form |
| endoscopic | SES-CD total score of 0-2 OR |
| remission | SES-CD total score of ≤4 and at least 2-point |
| | reduction from Baseline with no subscore > 1 |
| endoscopic | Minimum of 50% decrease from Baseline in |
| response | SES-CD total score |
| EQ-5D-5L | 5-level EuroQoL-5D |
| FACIT-F | Functional Assessment of Chronic Illness |
| | Therapy - Fatigue |
| FDA | Food and Drug Administration |
| FSH | follicle-stimulating hormone |
| GCP | Good Clinical Practice |
| HIV | human immunodeficiency virus |
| HRQoL | health-related quality of life |
| HRT | hormonal replacement therapy |
| IB | Investigator's Brochure |
| IBD | inflammatory bowel disease |
| IBDQ | Inflammatory Bowel Disease Questionnaire |
| ICF | informed consent form |
| ICH | International Council for Harmonisation |
| IEC | independent ethics committee |
| IFNγ | interferon-gamma |
| IL | interleukin |
| IRB | institutional review board |
| ITT | intent-to-treat |
| IV | intravenous |
| IWRS | interactive web response system |
| LCN2 | lipocalin 2 |
| LSF | loose stool frequency |
| MDRD | modification of diet in renal disease |
| MSP | medical safety physician |
| NCI | National Cancer Institute |
| NRS | Numeric Rating Scale |
| PCS | potentially clinically significant |
| PD | pharmacodynamics |
| PGIC-CD | Patient Global Impression of Change-Crohn's |
| | Disease |
| PGIS-CD | Patient Global Impression of Severity-Crohn's |
| | Disease |
| PII-LBMF | Patient Impression of Interference-Loose |
| | Bowel Movement Frequency |
| PIS-AP | Patient Impression of Severity-Abdominal Pain |
| PK | pharmacokinetics |
| Primary symptom | for participants with Baseline LSF subscore |
| remission | of ≥5 and AP subscore < 2: Average daily |
| | LSF subscore of ≤3 AND no worsening of |
| | Baseline AP subscore as assessed on the CDAI OR |
| | for participants with Baseline AP subscore |
| | of ≥2 and LSF subscore < 5: Average daily |
| | AP subscore of ≤1 AND no worsening of |
| | Baseline LSF subscore as assessed on the CDAI |
| PRO | patient-reported outcomes |
| PR | time from beginning of the P wave until the |
| | beginning of the QRS complex |
| QRS | time from beginning of the Q wave to end of |
| | the S wave in heart's electrical cycle |
| QT | time from beginning of the Q wave to end of |
| | the T wave in heart's electrical cycle |
| QTc | QT interval corrected for heart rate |
| QTcF | QT interval corrected for heart rate using |
| | the Fridericia formula (QTcF = QT/(RR)^{1/3}) |
| RNA | ribonucleic acid |
| SAE | serious adverse event |
| SAP | statistical analysis plan |
| SC | subcutaneously |
| SES-CD | Simple Endoscopic Score for Crohn's Disease |
| SF-36 | Short-Form 36 Health Survey |
| SoA | schedule of activities |
| TB | tuberculosis |
| TEAE | treatment-emergent adverse event |
| TESAE | treatment-emergent serious adverse event |
| TNFα | tumor necrosis factor-alpha |
| ULN | upper limit of normal |
| w/v | weight/volume |

### Example 2

Within the Phase 2b randomized control trial of Example 1 that compares the efficacy of Brazikumab to that of an active comparator in subjects with Crohn's disease it is expected that subjects with higher serum IL-22 (biomarker) levels will respond better to brazikumab therapy. This Example describes the modeling and criteria used to select an appropriate IL-22 cut-off value to define biomarker positive (BM+) versus negative (BM−) subpopulations. As noted above, in general the cut-off value will be determined in Stage 1 of the Phase 2b/3 study design. Exemplary cut-off values are expected to be in the range of about 9-50 pg/mL IL-22.

Efficacy of Brazikumab will be assessed based on two co-primary endpoints of achieving clinical PRO remission at 12 weeks (PRO-12) and achieving at least 50% endoscopic response at 12 weeks (ER₅₀-12).

Phase 2b will enroll all subjects regardless of IL-22 levels and randomize to the following four arms: (1) Brazikumab 1400 mg (1440 mg; Braz. High), (2) Brazikumab 700 mg (720 mg; Braz. Low), (3) Humira® 160 mg (AC), and (4) Placebo (PBO). There will be 125 subjects enrolled per arm for Braz. High, Braz. Low, and AC, and 75 subjects enrolled to PBO, resulting in 450 subjects total. A single primary final analysis will occur after full follow-up of the primary endpoint at 12 weeks. Subjects will continue to be followed for a maximum of 52 weeks to assess sustained response.

### REFERENCES

Ahern P P, Izcue A, Maloy K J, Powrie F. The interleukin-23 axis in intestinal inflammation Immunological Reviews. 2008; 226:147-159.
Andoh A, Zhang Z, Inatomi O, Fujino S, Deguchi Y, Araki Y, et al. Interleukin-22, a member of the IL-10 subfamily, induces inflammatory responses in colonic subepithelial myofibroblasts. Gastroenterology. 2005; 129:969-984.
Ben-Horin S, Kopylov U, Chowers Y. Optimizing anti-TNF treatments in inflammatory bowel disease. Autoimmunity Reviews, 2014; 13:24-30.
Best W R, Becktel J M, Singleton J W, Kern F Jr. Development of a Crohn's disease activity index. National Cooperative Crohn's Disease Study. Gastroenterology. 1976; 70:439-444.
Bettelli E, Korn T, Kuchroo V K. Th17: the third member of the effector T cell trilogy. Curr Opin Immunol. 2007; 19:652-657.
Brand S, Beigel F, Olszak T, Zitzmann K, Eichhorst S T, Otte J M, et al. IL-22 is increased in active Crohn's disease and promotes proinflammatory gene expression and intestinal epithelial cell migration. Am J Physiol Gastrointest Liver Physiol. 2006; 290:G827-G838.
Brazikumab (MEDI 2070) Investigator's Brochure, 2016; Edition 3.0.
Bretz F, Maurer W, Brannath W, Posch M. A graphical approach to sequentially rejective multiple test procedures. Statist. Med. 2009; 28:586-604
Burger D, Travis S. Conventional medical management of inflammatory bowel disease. Gastroenterology. 2011; 140:1827-1837.
Burton P R, Clayton D G, Cardon L R, Craddock N, Deloukas P, Duncanson A, et al. Association scan of 14,500 nonsynonymous SNPs in four diseases identifies autoimmunity variants. Nature Genet. 2007; 39:1329-1337.
Cargill M, Schrodi S J, Chang M, Garcia V E, Brandon R, Callis K P, et al. A large-scale genetic association study confirms IL12B and leads to the identification of IL23R as psoriasis-risk genes. Am J of Human Gen. 2007; 80:273-290.
Daperno M, D'Haens G, Van Assche G, Baert F, Bulois P, Maunoury V, et al. Development and validation of a new, simplified endoscopic activity score for Crohn's disease: the SES-CD. Gastrointest Endosc. 2004; 60:505-512.
Ding N S, Hart A, De Cruz P Aliment Pharmacol Ther. 2016 January; 43(1):30-51.
Duerr R H, Taylor K D, Brant S R, Rioux J D, Silverberg M S, Daly M J, et al. A genome-wide association study identifies IL23R as an inflammatory bowel disease gene. Science 2006; 314:1461-1463.
Farago B, Magyari L, Sáfrány E, Csöngei V, Járomi L, Horvatovich K, et al. Functional variants of interleukin-23 receptor gene confer risk for rheumatoid arthritis but not for systemic sclerosis. Ann Rheum Dis. 2008; 67:248-250.
Feagan B G, Sandborn W J, Gasink C, et al. Ustekinumab as induction and maintenance therapy for Crohn's disease. N Engl J Med 2016; 375:1946-1960.
Feagan B G, Sandborn W J, D'Haens G, Panés J, Kaser A, Ferrante M, et al. Induction therapy with the selective interleukin-23 inhibitor risankizumab in patients with moderate-to-severe Crohn's disease: a randomized, double-blind, placebo-controlled phase 2 study. Lancet. 2017; 389:1699-1709.
Gordon K, Langley R, Gottlieb, Papp K A, Krueger G G, Strober B E, et al. A phase III, randomized, controlled trial of the fully human IL-12/23 mAb briakinumab in moderate-to-severe psoriasis. J Invest Dermatol. 2012; 132:304-314.
Gottlieb A B; Leonardi C, Kerdel F, Mehlis S, Olds M, Williams D A. Efficacy and safety of briakinumab vs. etanercept and placebo in patients with moderate to severe chronic plaque psoriasis. Br J Dermatol. 2011; 165:652-660.
Guyatt G, Mitchell A, Irvine E J, Singer J, Williams N et al. A new measure of health status for clinical trials in inflammatory bowel disease. Gastroenterology. 1989 March; 96(3):804-810.
Higgins P D, Harding G, Patrick D L, Revicki D A, Globe G, Viswanathan H N, et al. Development of the Crohn's Disease Patient-Reported Outcomes (CD-PRO) Questionnaire. Gastroenterology. 2013; 44(5), Suppl 1, S-768 (Abstract Tu1124).
Hue S, Ahern P, Buonocore S, Kullberg M C, Cua D J, McKenzie B S, et al. Interleukin-23 drives innate and T cell-mediated intestinal inflammation. J Exp Med. 2006; 203:2473-2483.
Humira Package Insert. Abbvie Inc, North Chicago, Ill., USA.
Illes Z, Safrany E, Peterfalvi A, Magyari L, Farago B, Pozsonyi E, et al. 3′UTR C2370A allele of the IL-23 receptor gene is associated with relapsing-remitting multiple sclerosis. Neurosci Lett. 2008; 431:36-38.
Kimball A B, Papp K A, Wasfi Y, Chan D, Bissonnette R, Sofen H, et al. Long-term efficacy and safety of ustekinumab in patients with moderate to severe psoriasis through 5 years of follow-up: results from the PHOENIX 1 long-term extension. Br J Dermatol. 2012; 167(Suppl. 1):64(Abstract P94).
Kullberg, M C, Jankovic D, Feng C G, Hue S, Gorelick P L, McKenzie B S, et al. IL-23 plays a key role in Helicobacter hepaticus-induced T cell-dependent colitis. J Exp Med. 2006; 203:2485-2494.
Langley R G, Williams D, Papp K, Olds M. Long-term safety and efficacy of ABT-874 for the treatment of moderate to severe psoriasis: Interim analysis from an open-label extension study. J Am Acad Dermatol. 2012; 66:AB195 (Abstract 4779).
Lee E, Trepicchio W L, Oestreicher J L, Pittman D, Wang F, Chamian F, et al. Increased expression of interleukin 23 p19 and p40 in lesional skin of patients with psoriasis vulgaris. J Exp Med. 2004; 199:125-130.
Leonardi C L, Kimball A B, Papp K A, Yeilding N, Guzzo C, Wang Y, et al. Efficacy and safety of ustekinumab, a human interleukin-12/23 monoclonal antibody, in patients with psoriasis: 76-week results from a randomized, double-blind, placebo-controlled trial (PHOENIX 1). Lancet. 2008; 371:1665-1674.
Li Y, Chu N, Hu A, Gran B, Rostami A, Zhang G X. Increased IL-23p19 expression in multiple sclerosis lesions and its induction in microglia. Brain. 2007; 130:490-501.
Maca J, Bhattacharya S, Dragalin V, et al. Adaptive seamless Phase II/III designs—background, operational aspects, and examples. Drug Inf 2006; 40:463-475.
Mannon P J, Fuss I J, Mayer L, Elson C O, Sandborn W J, Present D, et al. Anti-interleukin 12 antibody for active Crohn's disease. N Eng J Med. 2004; 351:2069-2079.
Papp K A, Langley R G, Lebwohl M, Krueger G G, Szapary P, Yeilding N, et al. Efficacy and safety of ustekinumab, a human interleukin-12/23 monoclonal antibody, in patients with psoriasis: 52-week results from a randomized, double-blind, placebo-controlled trial (PHOENIX 2). Lancet. 2008; 371:1675-1684.
Reich K, Langley R G, Papp K A, Ortonne J-P, Unnebrink K, Kaul M, et al. A 52-week trial comparing briakinumab with methotrexate in patients with psoriasis. N Engl J Med. 2011; 365:1586-1596.
Rutgeerts P, Van Deventer S, Schreiber S. Review article: the expanding role of biological agents in the treatment of inflammatory bowel disease—focus on selective adhesion molecule inhibition. Aliment Pharmacol Ther. 2003; 17:1435-1450.
Sandborn W J, Gasink C, Gao L L, et al. Ustekinumab induction and maintenance therapy in refractory Crohn's disease. N Engl J Med 2012; 367: 1519-1528.
Sands B E, Ooi C J. A survey of methodological variation in the Crohn's disease activity index. Inflamm Bowel Dis. 2005; 11:133-138.
Sands B E, Chen J, Feagan B G, Penney M, Rees W A, Danese S, et al. Efficacy and safety of MEDI 2070, an antibody against interleukin 23, in patients with moderate to severe Crohn's disease: a phase 2a study. Gastroenterology 2017; 153:77-86.
Schmidt C, Giese T, Ludwig B, Mueller-Molaian I, Marth T, Zeuzem S, et al. Expression of interleukin-12-related cytokine transcripts in inflammatory bowel disease: elevated interleukin-23p19 and interleukin-27p28 in Crohn's disease but not in ulcerative colitis. Inflamm Bowel Dis. 2005; 11:16-23.
Sofen H, Smith S, Matheson R, Leonardi C, Calderon C, Bouman E, et al. Results of a single ascending dose study to assess the safety and tolerability of CNTO 1959 following intravenous or subcutaneous administration in healthy subjects and in subjects with moderate to severe psoriasis. Br J Dermatol. 2011; 165:e10 (Abstract FC-21).
Strober B E, Crowley J J, Yamauchi P S, Olds M, Williams D A. Efficacy and safety results from a phase III, randomized controlled trial comparing the safety and efficacy of briakinumab with etanercept and placebo in patients with moderate to severe chronic plaque psoriasis. Br J Dermatol. 2011; 165:661-668.
Tinsley A, Macklin E A, Korzenik J R, Sands B E. Validation of the functional assessment of chronic illness therapy-fatigue (FACIT-F) in patients with inflammatory bowel disease. Aliment Pharmacol Ther. 2011; 34:1328-1336.
Uhlig H H, McKenzie B S, Hue S, Thompson C, Joyce-Shaikh B, Stepankova R, et al. Differential activity of IL-12 and IL-23 in mucosal and systemic innate immune pathology. Immunity. 2006; 25:309-318.
Vaknin-Dembinsky A, Balashov K, Weiner H L. IL-23 is increased in dendritic cells in multiple sclerosis and down regulation of IL-23 by antisense oligos increases dendritic cell IL-10 production. J of Immunol. 2006; 176:7768-7774.
Yen D, Cheung J, Scheerens H, Poulet F, McClanahan T, McKenzie B, et al. IL-23 is essential for T cell-mediated colitis and promotes inflammation via IL-17 and IL-6. J Clin Invest. 2006; 116:1310-1316.

All patents and other publications identified are expressly incorporated herein by reference in their entirety for the purpose of describing and disclosing, for example, the methodologies described in such publications that might be used in connection with information described herein.

1. A method of treating Crohn's disease in a subject comprising administration of a plurality of intravenous doses of an anti-IL-23 antibody to the subject followed by administration of a plurality of subcutaneous doses of the anti-IL-23 antibody to the subject; wherein:
each intravenous dose comprises 720 mg or 1440 mg of the anti-IL23 antibody;
each subcutaneous dose comprises 240 mg of the anti-IL23 antibody; and
the anti-IL23 antibody has:
(a) a heavy chain variable region comprising Complementarity Determining Regions (CDRs) with the following amino acid sequences: (i) CDR1: SYGMH (SEQ ID NO:3), (ii) CDR2: VIWYDGSNEYYADSVKGR (SEQ ID NO:4), and (iii) CDR3: DRGYTSSWYPDAFDI (SEQ ID NO:5); and
(b) a light chain variable region comprising CDRs with the following amino acid sequences: (i) CDR1: TGSSSNTGAGYDVH (SEQ ID NO:6), (ii) CDR2: GSGNRPS (SEQ ID NO:7), and (iii) CDR3: QSYDSSLSGWV (SEQ ID NO:8).

2. The method of claim 1 wherein a biological sample of the subject has an IL-22 level of at least about 9 pg/ml or at least about 50 pg/ml.

3. The method according to claim 1, wherein each intravenous dose comprises 720 mg of the anti-IL23 antibody.

4. The method according to claim 1, wherein each intravenous dose comprises 1440 mg of the anti-IL23 antibody.

5. The method according to claim 1, wherein the method comprises administration of three intravenous doses of the anti-IL23 antibody to the subject.

6. (canceled)

7. The method of claim 5 wherein the method comprises administration of intravenous doses to the subject on days 1, 29 and 57 of treatment.

8. The method of claim 1 wherein the plurality of subcutaneous doses are administered to the subject at least 12 weeks after initiating treatment.

9. The method of claim 8, wherein the subcutaneous administrations are delivered doses are administered on about day 85 of treatment and about every 4 weeks thereafter.

10-11. (canceled)

12. The method of claim 1 wherein the anti-IL-23 antibody is administered to the subject in an amount and at an interval of:
(a) intravenous doses comprising 720 mg or 1440 mg of the anti-IL23 antibody on or about days 1, 29, and 57 of treatment, followed by
(b) subcutaneous doses comprising 240 mg of the anti-IL23 antibody on or about day 85 of treatment and about every 4 weeks thereafter through at least week 48.

13. (canceled)

14. The method of claim 1 wherein the anti-IL-23 antibody is Brazikumab. cm 15-16. (canceled)

17. The method of claim 14 wherein 1440 mg of Brazikumab is administered intravenously on days 1, 29 and 57.

18. The method of claim 14 wherein 720 mg of Brazikumab is administered intravenously on days 1, 29 and 57.

19. The method of claim 14 wherein 240 mg of Brazikumab is administered subcutaneously on or about day 85 and about every 4 weeks thereafter through at least week 48.

20. The method of claim 19 wherein 240 mg of Brazikumab is administered subcutaneously on day 85 and about every 4 weeks thereafter through at least week 48.

21. The method of claim 20 wherein 240 mg of Brazikumab is administered subcutaneously on day 85 and every 4 weeks thereafter through at least week 48.

22. The method of claim 21 wherein 240 mg of Brazikumab is administered subcutaneously on day 85 and every 4 weeks thereafter through week 48-52.

23. The method of claim 22 wherein 240 mg of Brazikumab is administered subcutaneously on day 85 and every 4 weeks thereafter through week 48.

24. A method of selecting a subject amenable to treatment of Crohn's disease with Brazikumab comprising
(a) obtaining a biological sample from the subject;
(b) measuring the level of IL-22 in the sample;
(c) comparing the level of IL-22 in the sample to the level of IL-22 in a control; and
(d) selecting the subject as amenable to treatment of Crohn's disease with Brazikumab if the level of IL-22 is higher in the sample than in the control.

25-34. (canceled)

35. A method of identifying a subject as a member of a patient sub-population amenable to treatment for Crohn's disease with Brazikumab comprising
(a) obtaining a biological sample from the subject;
(b) measuring the level of IL-22 in the sample;
(c) comparing the level of IL-22 in the sample to the level of IL-22 in a control; and
(d) identifying the subject as a member of the patient sub-population amenable to treatment for Crohn's disease with Brazikumab if the level of IL-22 in the subject's sample is higher than in the control.

36-45. (canceled)

46. The method according to claim 8, wherein subsequent subcutaneous doses are administered about every 4 weeks after the first subcutaneous dose.

47. The method according to claim 9, wherein subcutaneous administration continues through at least week 48 of treatment.

48. The method according to claim 5, wherein each intravenous dose comprises 720 mg of the anti-IL23 antibody.

49. The method according to claim 5, wherein each intravenous dose comprises 1440 mg of the anti-IL23 antibody.

50. The method according to claim 1, wherein:
the method comprises administration of three intravenous doses of Brazikumab to the subject;
each intravenous dose comprises 720 mg of Brazikumab; and
the plurality of subcutaneous doses are administered to the subject at least 12 weeks after initiating treatment.

51. The method according to claim 50, wherein subsequent subcutaneous doses are administered about every 4 weeks after the first subcutaneous dose.

52. The method according to claim 51, wherein subcutaneous administration continues through at least week 48 of treatment.

53. The method according to claim 1, wherein:
the method comprises administration of three intravenous doses of Brazikumab to the subject;
each intravenous dose comprises 1440 mg of Brazikumab; and
the plurality of subcutaneous doses are administered to the subject at least 12 weeks after initiating treatment.

54. The method according to claim 53, wherein subsequent subcutaneous doses are administered about every 4 weeks after the first subcutaneous dose.

55. The method according to claim 54, wherein subcutaneous administration continues through at least week 48 of treatment.
